SEQUENCE LISTING (24 sequences)

SEQ ID NO: 1 - DNA, 21 bp, Artificial Sequence
  IL-6 Mouse Primer Sequence (Forward)
gaacaacgatgatgcacttgc

SEQ ID NO: 2 - DNA, 21 bp, Artificial Sequence
  IL-6 Mouse Primer Sequence (Reverse)
tccaggtagctatggtactcc

SEQ ID NO: 3 - DNA, 20 bp, Artificial Sequence
  IL-1b Mouse Primer Sequence (Forward)
ctccacctcaatggacagaa

SEQ ID NO: 4 - DNA, 20 bp, Artificial Sequence
  IL-1b Mouse Primer Sequence (Reverse)
gccgtctttcattacacagg

SEQ ID NO: 5 - DNA, 20 bp, Artificial Sequence
  TNC Mouse Primer Sequence (Forward)
atcccttcatcagcagtcca

SEQ ID NO: 6 - DNA, 20 bp, Artificial Sequence
  TNC Mouse Primer Sequence (Reverse)
gcatccgtaccaaaaccatc

SEQ ID NO: 7 - DNA, 19 bp, Artificial Sequence
  CDK1 Mouse Primer Sequence (Forward)
cagagattgaccagctctt

SEQ ID NO: 8 - DNA, 20 bp, Artificial Sequence
  CDK1 Mouse Primer Sequence (Reverse)
gaaaggtgttcttgtagtcc

SEQ ID NO: 9 - DNA, 22 bp, Artificial Sequence
  TOP2A Mouse Primer Sequence (Forward)
cggaatgacaagcgagaagtaa

SEQ ID NO: 10 - DNA, 24 bp, Artificial Sequence
  TOP2A Mouse Primer Sequence (Reverse)
gcattgtaaagatgtatcgtggac

SEQ ID NO: 11 - DNA, 22 bp, Artificial Sequence
  Mki67 Mouse Primer Sequence (Forward)
aatccaactcaagtaaacgggg

SEQ ID NO: 12 - DNA, 20 bp, Artificial Sequence
  Mki67 Mouse Primer Sequence (Reverse)
ttggcttgcttccatcctca

SEQ ID NO: 13 - DNA, 22 bp, Artificial Sequence
  GAPDH Mouse Primer Sequence (Forward)
tggcaaagtggagattgttgcc

SEQ ID NO: 14 - DNA, 21 bp, Artificial Sequence
  GAPDH Mouse Primer Sequence (Reverse)
aagatggtgatgggcttcccg

SEQ ID NO: 15 - DNA, 20 bp, Artificial Sequence
  hIL1b Human Primer Sequence (Forward)
gaccttccaggatgaggaca

SEQ ID NO: 16 - DNA, 20 bp, Artificial Sequence
  hIL1b Human Primer Sequence (Reverse)
agctcatatgggtccgacag

SEQ ID NO: 17 - DNA, 20 bp, Artificial Sequence
  hIL6 Human Primer Sequence (Forward)
ggtacatcctcgacgtgtct

SEQ ID NO: 18 - DNA, 21 bp, Artificial Sequence
  hIL6 Human Primer Sequence (Reverse)
gtgcctctttgctgctttcac

SEQ ID NO: 19 - DNA, 20 bp, Artificial Sequence
  hCOL1A2A Human Primer Sequence (Forward)
ccgtgcttctcagaagacag

SEQ ID NO: 20 - DNA, 20 bp, Artificial Sequence
  hCOL1A2A Human Primer Sequence (Reverse)
cttgccccattcattcatca

SEQ ID NO: 21 - DNA, 20 bp, Artificial Sequence
  hCCL2 Human Primer Sequence (Forward)
ccccagtcacctgctgttat

SEQ ID NO: 22 - DNA, 20 bp, Artificial Sequence
  hCCL2 Human Primer Sequence (Reverse)
tggaatcctgaacccacttc

SEQ ID NO: 23 - DNA, 20 bp, Artificial Sequence
  hHPRT Human Primer Sequence (Forward)
atgggaggccatcacattgt

SEQ ID NO: 24 - DNA, 22 bp, Artificial Sequence
  hHPRT Human Primer Sequence (Reverse)
atgtaatccagcaggtcagcaa

(19) United States
(12) Patent Application Publication
(10) Pub. No.: US 2023/0137092 A1
(43) Pub. Date: May 4, 2023

(54) Composition and Method for the Treatment and Prevention of Cardiac, Pulmonary, Dermal, and Renal Fibrosis

(71) Applicant: EMERALD HEALTH PHARMACEUTICALS INC., San Diego, CA (US)

(72) Inventors: Alain ROLLAND, San Diego, CA (US); Eduardo MUÑOZ-BLANCO, Córdoba (ES); Adela GARCIA-MARTIN, Córdoba (ES); Carmen NAVARRETE, Córdoba (ES)

(73) Assignee: EMERALD HEALTH PHARMACEUTICALS INC., San Diego, CA (US)

(21) Appl. No.: 17/760,283

(22) PCT Filed: Feb. 8, 2021
(86) PCT No.: PCT/US2021/017052

§ 371 Date: Aug. 5, 2022

Related U.S. Application Data
(60) Provisional application No. 63/020,584, filed on May 6, 2020.
(60) Provisional application No. 62/970,854, filed on Feb. 6, 2020.

(51) Int. Cl. A61K 31/122; A61K 31/164; A61P 9/00

(52) U.S. Cl. CPC A61K 31/122; A61K 31/164; A61P 9/00

(57) ABSTRACT

The present disclosure relates to methods of treating or preventing various diseases or disorders, such as cardiac fibrosis, aortic fibrosis, pulmonary fibrosis, renal fibrosis, dermal fibrosis, and chronic kidney diseases, using cannabidiol derivatives or compositions thereof.

### CROSS REFERENCE TO RELATED APPLICATIONS

This application claims priority to and the benefit of U.S. Application No. 62/970,854, filed Feb. 6, 2020, and U.S. Application No. 63/020,584, filed May 6, 2020, the disclosures of which are hereby incorporated by reference herein in their entireties.

### FIELD OF THE DISCLOSURE

The present disclosure relates to compositions comprising at least one cannabidiol derivative and methods for prevention or treatment of various diseases and disorder, such as cardiac, aortic, renal, pulmonary, and dermal fibrotic diseases or disorders, using said cannabidiol derivatives or compositions thereof. In some embodiments, the present disclosure relates to methods of preventing or treating cardiac, aortic, renal, pulmonary, and dermal fibrotic diseases or disorders, using cannabidiol aminoquinone derivatives or compositions thereof.

### BACKGROUND OF THE DISCLOSURE

Heart failure, the clinical manifestation of numerous forms of cardiovascular disease, is a devastating disorder characterized by interstitial fibrosis, chamber remodeling, and reduced ventricular compliance. Heart disease remains the predominant cause of mortality in in the world (Benjamin E J et al., 2019, Circulation, 139: e56-e528). Despite substantial improvements in therapeutic strategies, cardiovascular disease remains the leading cause of death worldwide indicating an urgent need for innovative treatment strategies (Mozaffarian D et al., 2015, Circulation, 131: e29-e322). Nearly all forms of heart disease including myocardial infarction, hypertension, atrial fibrillation, diabetes mellitus and several other cardiovascular diseases are associated with cardiac fibrosis.

Cardiac fibrosis is characterized by abnormal accumulation of extracellular matrix (ECM) in the myocardium (Berk B C et al., 2007, J. Clin. Invest., 117:568-5751; Kong Pet al., 2014, Cell Mol. Life Sci., 71:549-574) accompanied by the activation of the cardiac fibroblast (Krenning G et al., 2010, J. Cell Physiol., 225: 631-637) both are integral components of most cardiac pathologic conditions. The net accumulation, abolishes cardiac contractility, impairs cardiac function and ultimately leads to heart failure. Because the adult mammalian myocardium has negligible regenerative capacity, the most extensive fibrotic remodeling of the ventricle is found in diseases associated with acute cardiomyocyte death. Following acute myocardial infarction, sudden loss of a large number of cardiomyocytes triggers an inflammatory reaction, ultimately leading to replacement of dead myocardium with a collagen-based scar (Frangogiannis N G, 2012, Circ. Res., 110:159-173). Several other pathophysiologic conditions induce more insidious interstitial and perivascular deposition of collagen, in the absence of completed infarction. Aging is associated with progressive fibrosis that may contribute to the development of diastolic heart failure in elderly patients. Pressure overload, induced by hypertension or aortic stenosis, results in extensive cardiac fibrosis that is initially associated with increased stiffness and diastolic dysfunction and systolic heart failure (Berk B C et al., 2007, J. Clin. Invest., 117:568-5751). Volume overload due to valvular regurgitant lesions may also result in cardiac fibrosis, characterized by disproportionately large amounts of non-collagenous matrix (Borer J S et al., 2002, Circulation, 105:1837-1842).

Cardiac fibroblasts are the main effector cells in cardiac fibrosis, and play an important role in the formation of cardiac fibrosis because it is the principle cell type implicated in the critical phases of the fibrotic process. Its response consists of inflammation, proliferation of non-myocytes, scar maturation, production of excessive synthesis, and accumulation of ECM proteins, including collagen deposition in cardiac tissue (Travers et al., 2016, Circ. Res., 118:1021-1040).

The expression of various pro-inflammatory cytokines and pro-fibrotic factors is upregulated in cardiac fibroblasts, which can be transformed into myofibroblasts that exhibit augmented proliferative, migratory, contractile, and collagen producing abilities (Krenning G et al., 2010, J. Cell. Physiol., 225: 631-637; Ivey M J et al., 2016, Circ. J., 80:2269-2276).

Suppression of proliferation and induction of apoptosis reduce activated fibroblasts and thus alleviate cardiac fibrosis.

It has been demonstrated that the modulation of cyclin-dependent kinase 1 (CDK1), a known target of the E2 factor (E2F) pathway, is involved in G1 checkpoint regulation, inhibits of the proliferation of cardiac fibroblast and exerts a protective effect on cardiac fibrosis (Fang Get al., 2018, Molecular Medicine Reports, 18:1433-1438). The E2F family of transcription factors play crucial roles in controlling the expression of genes involved in cell proliferation, differentiation, and apoptosis. In fact, they play a pivotal role in the G1/S phase transition of the cell cycle (Dyson N., 1998, Genes Dev., 12:2245-2262; Nevins J R et al., 1997, J. Cell. Physiol., 173:233-236).

Structurally and functionally, the E2F transcription factors can be divided into three main categories as follows: E2Fs 1-3, which are involved in proliferation; E2F-4 and E2F-5 that are thought to play a role in differentiation; and E2F-6, which is described as a transcriptional repressor (Trimarchi, J M et al., 2002, Nat. Rev. Mol. Cell Biol., 3:11-20). For full transcriptional activity, the E2F transcription factors heterodimerize with a dimerization partner (DP) protein, of which two mammalian forms have been described, namely DP-1 and DP-2 (Rogers K T et al., 1996, Proc. Natl. Acad. Sci. U.S.A., 93:7594-75998). The transcriptional activity of E2F members is sterically regulated by the pRb family of pocket proteins that consists of pRb, p107, and p130 (Dyson N, 1998, Genes Dev., 12:2245-2262; Rogers K T et al., 1996, Proc. Natl. Acad. Sci. U.S.A., 93:7594-75998). E2F activation occurs when cyclin D-cyclin-de-pendent kinase (CDK) 4/6 complexes in the G1 phase of the cell cycle phosphorylate the pRb pocket proteins, which then dissociate from the E2F-DP complex, resulting in E2F-mediated gene transcription and progression to the S phase of the cell cycle (Dyson N, 1998, Genes Dev., 12:2245-2262; Nevins J R et al., 1997, J. Cell. Physiol., 173:233-236).

Various studies have shown that E2F is instrumental in cardiac myocyte cell cycle progression (Flink I et al., 1998, J. Mol. Cell. Cardiol., 30:563-578). E2F activity is increased during the development of myocyte hypertrophy and blocking E2F function inhibits the development of cardiomyocytes hypertrophy (Vara D et al., 2003, J. Biol. Chem., 278:21388-21394). The dead myocytes are replaced with proliferating cardiac fibroblasts that synthesize collagen matrix.

Although some conventional therapies, such as renin-angiotensin-aldosterone system inhibitors (i.e., Losartan) reduce cardiac fibrosis in humans, cardiac fibrosis continues to persist in patients with heart failure even when treated with these conventional therapies, indicating a need to develop novel and effective anti-fibrotic therapies in cardiovascular disease (Fang Let al., 2017, Front. Pharmacol., 8:186).

Idiopathic pulmonary arterial hypertension (iPAH) is a fatal disease with an average mortality rate of 58% within 3 years after diagnosis. It is well known that the renin-angiotensin system plays an important role in endothelial function, dysfunction and vascular remodeling. The activation of the classical angiotensin-converting enzyme (ACE)-angiotensin-II (Ang-II)-angiotensin 1 receptor (AT1R) axis of the RAS (ACE-Ang II-AT1R) adversely affects pulmonary hemodynamics to cause vasoconstriction and pulmonary hypertension (Marshall R P, 2003, Curr. Pharm. Des., 9:715-722). There is evidence that this part of the RAS axis with elevated ACE expression and Ang-II production as well as increased AT1R expression can occur in patients with pulmonary arterial hypertension (PAH) (de Man F S et al., 2012, Am. J. Respir. Crit. Care Med.; 186:780-789). PAH is also associated with a particularly severe arteriopathy and vascular lesions (e.g., plexiform lesion and neointimal proliferation), which obstruct the pulmonary arteries and arterioles. PAH is a common finding in patients with aortic stenosis and has been associated with increased mortality and morbidity following both transcatheter aortic valve replacement and surgical aortic valve replacement. (O'Sullivan C J et al., 2015, Circ. Cardiovasc. Interv. 8:e002358).

In addition, PAH is a well-recognized complication of interstitial lung disease, including idiopathic pulmonary fibrosis (IPF). The underlying pathogenesis was initially hypothesized to be inflammatory but now is characterized as an over exuberant fibroproliferative process (Shorr A F et al., 2007, Eur. Respir. J., 30:715-721). Indeed, PAH is commonly present in patients with chronic lung diseases, such as chronic obstructive pulmonary disease (COPD). PAH has been identified to be present in as much as 40% of patients with COPD or IPF and it is considered as one of the principal predictors of mortality in patients with COPD or IPF. COPD is a complex disorder that primarily affects the lungs and is characterized not only by local pulmonary, but also by systemic inflammation, which promotes the development of extrapulmonary and cardiovascular co-morbidities. ACE inhibitors and ARBs (angiotensin receptor blockers) are widely used drugs in the treatment of cardiovascular diseases, with growing evidence suggesting potential benefits in COPD patients. However, the effect of such type of drugs is limited and despite the prevalence and fatal consequences of PAH in the setting of chronic lung diseases is not decreasing. Thus, there are limited therapies available for patients with advanced diseases, with lung transplantation remaining as the most viable option.

Furthermore, chronic kidney disease is a leading cause of end stage renal disease and cardiovascular morbidity and mortality worldwide, resulting in a growing social and economic burden. The prevalence and burden of chronic kidney disease is anticipated to further increase over the next decades as a result of aging. Kidney fibrosis is a major hallmark of chronic kidney disease that is a progressive pathophysiological change that is associated with damage and functional loss of the kidney.

Biological meaning of fibrosis during the progression of chronic kidney diseases depends on renal myofibroblasts contributing to ECM production (Mack M et al., 2015, Kidney Int., 87:297-307). Under normal conditions, resident renal fibroblasts produce erythropoietin (EPO), a hormone best known as a regulator of red blood cell production, in response to hypoxic insults to maintain physiological homeostasis. However, under pathologic conditions, the resident renal fibroblasts transdifferentiate into myofibroblasts, which promote renal fibrosis by producing large amounts of ECM proteins rather than EPO (Sato Y et al, 2017, Inflamm Regen., 37:17). Moreover, one study in mice showed that treatment with EPO inhibited the accumulation of fibrocyte by inhibition of α-SMA upregulation, attenuating renal interstitial fibrosis (Geng X C et al., 2015, Mol. Med. Rep., 11:3860-3865). Thus, EPO provides efficient protection against renal fibrosis (Zhang Y et al., 2020, Front. Med. (Lausanne), 7:47).

In patients with chronic kidney disease (CKD), kidney function is severely compromised. CKD, also known as chronic renal disease, is a progressive loss in renal function over a period of months or years. The most severe stage of CKD is End Stage Renal Disease (ESRD), which occurs when the kidneys cease to function. The two main causes of CKD are diabetes and high blood pressure, which are responsible for up to two-thirds of the cases. Heart disease is the leading cause of death for all people having CKD. Excessive fluid can accumulate in patients suffering from ESRD. The mortality rate of ESRD patients who receive traditional hemodialysis therapy is 24% per year with an even higher mortality rate among diabetic patients. Fluid accumulates in ESRD patients because the kidneys can no longer effectively remove water and other fluids from the body. The fluid accumulates first in the blood and then accumulates throughout the body, resulting in swelling of the extremities and other tissues as edema. This accumulation of fluid causes increased stress on the heart, in turn causing significant increases in blood pressure or hypertension, which can lead to heart failure.

Although the population of patients afflicted with CKD grows each year, there is no cure. Current treatments for CKD seek to slow the progression of the disease. However, as the disease progresses, renal function decreases, and, eventually, renal replacement therapy is employed to compensate for lost kidney function. Renal replacement therapy entails either transplantation of a new kidney or dialysis.

For kidney diseases, inhibitors of renin-angiotensin-aldosterone system directly exert antifibrotic activity (Tampe D et al., 2014, Nat. Rev. Nephrol., 10,226-237). However, since the ACE inhibitor captopril was approved by the FDA in 1981, incidence of end stage renal disease (ESRD) has more than tripled in the USA. (Collins A J et al., 2011, Am. J. Kidney Dis., 59:evii). However, there is currently no medication on the market to prevent or treat cardiac or renal fibrosis.

Systemic lupus erythematosus (SLE) is a chronic autoimmune disease that is typically characterized by the formation of autoantibodies, such as antinuclear antibodies and anti-double-stranded DNA antibodies. SLE has diverse clinical presentations involving multiple organs, including kidneys. Renal involvement is one of the most frequent and serious complications of SLE, and the presentation varies from subclinical laboratory abnormalities to overt nephritis or nephrotic syndrome. Furthermore, SLE is one of the major causes of CKD and ESRD. It is known that renal involvement occurs in approximately 60% of SLE patients, and 10% to 20% of them progress to ESRD. CKD and ESRD caused by lupus nephritis are major causes of mortality in SLE patients. Although there have been recent advances in immune-suppressive treatment, progression to ESRD and mortality has not declined in the last decades (Costenbader K H et al., 2011, Arthritis Rheum., 63:1681-1688). In addition, mid-wall myocardial fibrosis occurs frequently in SLE and is strongly associated with advancing subject age, but not with SLE duration or severity (Seneviratne M G et al., 2016, Lupus, 25:573-581). Therefore, novel drug candidates aimed to prevent and/or treat cardiac, pulmonary, and renal fibrosis and endowed with immunomodulatory activities are required for the treatment of SLE.

Available evidence indicates that cystic fibrosis (CF) causes widespread involvement of the cardiovascular system. Aside from the heart, unusual aberrations have been observed in the bronchial arteries, the aorta, and the systemic capillaries. Of all cardiovascular complications, cor pulmonale is the most serious. The basis of cor pulmonale is hypoxemia and unless this is relieved, no enduring effect can be expected from therapy directed toward the heart. Inasmuch as cystic fibrosis is a progressive disease, cor pulmonale is also progressive. The lung involvement is usually progressive with intermittent exacerbations. Aggressive management and advances in treatment delay but do not prevent progression of lung disease. Pulmonary hypertension correlates with the degree of hypoxemia. Pulmonary hypertension is common in individuals with advanced CF-related lung disease. In addition, accumulating data from pathological, animal, and human studies have shown that cystic fibrosis results in cardiac functional and structural changes, independent of any other known cardiac disease; which supports the existence of cystic fibrosis related cardiomyopathy. Histologic studies have demonstrated that myocardial fibrosis could affect early in life the left ventricle (Labombarda F et al, 2016, Respir Med., 118:31-38).

On the other hand, CF is one of the success stories of modern medicine—whereas in the 1950s most individuals with the disease died before the age of five, medical advances mean that those born in the 21st century will live into their fifth or sixth decade. However, the aging of the CF population has brought with it unforeseen problems and complications, and there has been a paradigm shift in outlook in the adult healthcare sector; from a focus on the care of lung disease to the management of a complex multi-system chronic illness. Thus, CF individuals are in danger of acute kidney injury and the development of chronic renal disease through exposure to multiple potentially nephrotoxic agents, including aminoglycosides, non-steroidal anti-inflammatory drugs (NSAIDs), and immune-suppressants (Nazareth D et al., 2013, J. Cyst. Fibros., 12:309-317).

Dermatomyositis (DM) is a distinctive systemic connective tissue disease whereby the skin defines a cardinal site of involvement. Evidence indicates that a significant component of its clinical manifestations may be related to endothelial cell injury. The identification of fibrosis in biopsies of skin lesions typical clinically for DM has been attributed to severe autoimmune-based endothelial cell injury phenomenon (Magro C M et al., 2018, J. Cutan. Pathol., 35:31-39).

Myelin sheaths, which cover many nerve fibers, are composed of lipoprotein layers formed in early life. Myelin formed by the oligodendroglia in the central nervous system (CNS) differs chemically and immunologically from that formed by the Schwann cells peripherally, but both types have the same function: to promote transmission of a neural impulse along an axon. Many congenital metabolic disorders (e.g., phenylketonuria and other aminoacidurias; Tay-Sachs, Niemann-Pick, and Gaucher's diseases; Hurler's syndrome; Krabbe's disease and other leukodystrophies) affect the developing myelin sheath, mainly in the CNS. Unless the biochemical defect can be corrected or compensated for, permanent, often widespread, neurologic deficits result.

Demyelination in later life is a feature of many neurologic disorders; it can result from damage to nerves or myelin due to local injury, ischemia, toxic agents, or metabolic disorders. Extensive myelin loss is usually followed by axonal degeneration and often by cell body degeneration, both of which may be irreversible. However, remyelination occurs in many instances, and repair, regeneration, and complete recovery of neural function can be rapid. Recovery often occurs after the segmental demyelination that characterizes many peripheral neuropathies; this process may account for the exacerbations and remissions of MS. Central demyelination (i.e., of the spinal cord, brain, or optic nerves) is the predominant finding in primary demyelinating diseases, whose etiology is unknown. The most well-known is MS. Other diseases include, for example, acute disseminated encephalomyelitis (postinfectious encephalomyelitis), adrenoleukodystrophy, adrenomyeloneuropathy, Leber's hereditary optic atrophy and related mitochondrial disorders and human T-cell lymphotropic virus (HTLV) infection-associated myelopathy.

Remyelination is generally accepted as a regular event in MS lesions; however, it is insufficient for myelin repair and axons remain demyelinated in MS patients. Possible explanations for this include failure of recruitment or survival of oligodendrocyte progenitor cells (OPCs), disturbance of differentiation/maturation of OPCs, and loss of capability of myelin forming. Therefore, effective interventions for MS should not only prevent disease progression, but also promote remyelination.

Thus, there remains a need in the art for clinical interventions and therapies that target kidney fibrosis as well as medication that prevent or treat cardiac, aortic, dermal, and pulmonary fibrosis. There is also a need in the art for a disease-modifying drug, and a composition thereof that targets complementary signaling pathways that alleviate neuroinflammation and favor both neuroprotection and myelin regeneration for management and treatment of various autoimmune diseases, demyelinating diseases, inflammatory-related disorders, cardiac diseases or disorders, cardiovascular diseases or disorders, and diseases of the central nervous system (CNS). There also remains a need in the art for development of efficacious pharmaceutical preparation. The present disclosure addresses these needs.

### SUMMARY OF THE DISCLOSURE

The disclosure provides compositions comprising at least one cannabidiol derivative solubilized in a pharmaceutical vehicle. In one aspect, the compositions have increased bioavailability. In another aspect, the compositions have increased solubility.

In another aspect, disclosed herein are methods for preventing or treating a disease or disorder in a subject in need thereof, wherein the method comprises administering a therapeutically amount of a compound having the structure of Formula (I) or a composition thereof to a subject in need thereof,

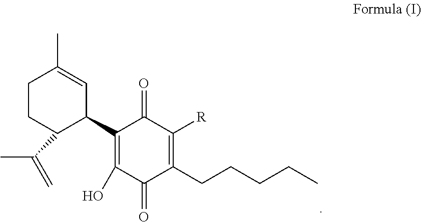

In some embodiments, R is the nitrogen atom of a group independently selected from a linear or branched alkylamine, an arylamine, an arylalkylamine, a heteroarylamine, a heteroarylalkylamine, a linear or branched alkenylamine, a linear or branched alkynylamine, or NH₂.

In some embodiments, the cannabidiol derivatives disclosed herein are selected from one or more of the following compounds:

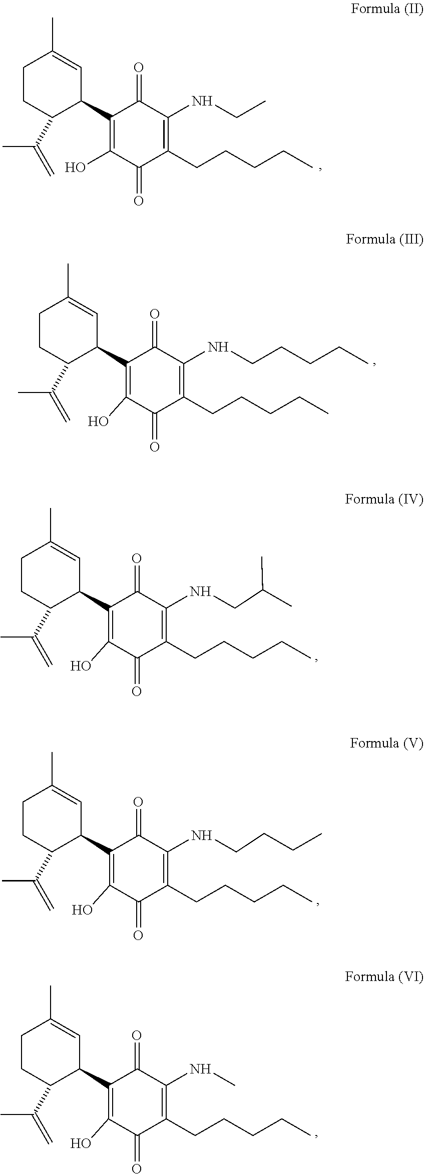

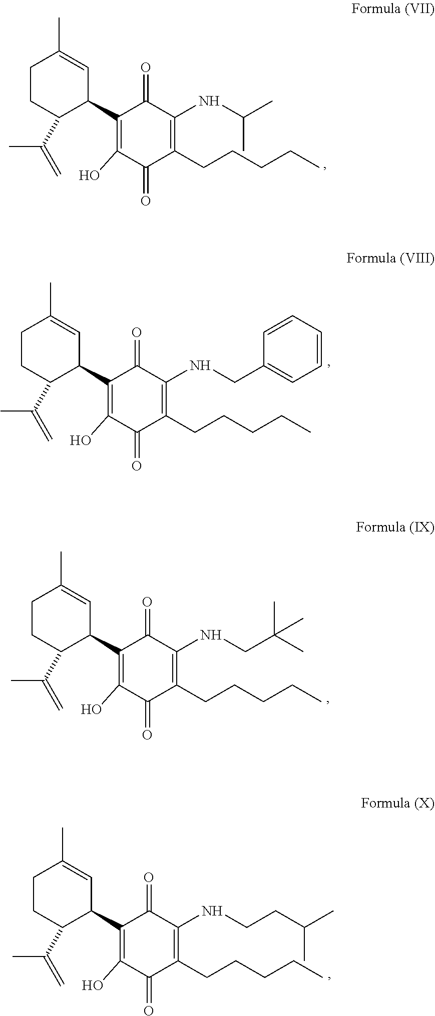

and any combination thereof.

In another aspect, the disclosure relates to methods of treating a condition or disease by administering one or more compounds disclosed herein such as those having the structure of Formula (I)-(X) or a composition thereof. In some embodiments, administering one or more compounds disclosed herein such as those having the structure of Formula (I)-(X) or a composition thereof modulates and/or inhibits one or more of more of the activity of E2F pathway, level of myofibroblasts, level of fibroblasts, level of fibrosis, level of macrophage infiltration, level of collagen accumulation or deposition, aortic media thickness, aortic media thickness associated with fibrosis, level or activity of inflammatory response, level or activity of at least one fibrotic protein, level or activity of interferon response, level or activity of interferon α (IFN-α) response, level or activity of interferon γ (IFN-γ) response, immune response associated with fibrosis, autoimmune response associated with fibrosis, inflammation associated with fibrosis, or any combination thereof.

In another embodiment, the treated condition or disease is further responsive to the modulation of the CB₂ activity. In one embodiment, the condition or disease responsive to the modulation of the E2F transcriptional signature is further responsive to the modulation of the CB₂ activity.

In some embodiments, the collagen accumulation or deposition is one or more of interstitial collagen accumulation or deposition, interstitial collagen accumulation or deposition in cardiac tissue, myocardial collagen accumulation or deposition, perivascular myocardial collagen accumulation or deposition, renal collagen accumulation or deposition, dermal collagen accumulation or deposition, pulmonary collagen accumulation or deposition, or any combination thereof.

In some embodiments, the compound of having the structure of Formula (I)-(X) or a composition thereof modulates at least one or more genes selected from a E2F-dependent gene, cyclin-dependent kinase 1 (CDK1) gene, topoisomerase 2-alpha (TOP2A) gene, Proliferation Ki-67 (MKi67) gene, inflammatory gene, profibrotic gene, Interleukin 6 (IL6) gene, Interleukin 1β (IL1β) gene, tenascin-C (TNC) gene, or any combination thereof.

In some embodiments, the fibrosis is a renal fibrosis, dermal fibrosis, pulmonary fibrosis, cardiac fibrosis, aortic fibrosis or disorder, or any combination thereof.

In some embodiments, the compounds disclosed herein are used to treat or prevent a disease or condition associated with the E2F pathway, such as a disease or condition associated with proliferation of myofibroblast; a disease or condition associated with collagen accumulation or deposition; a disease or condition associated with increased aortic media thickness; a disease or condition associated with aortic media thickness by fibrosis; a disease or condition associated with inflammation; a disease or condition associated with macrophage infiltration; a disease or condition associated with fibrotic proteins expression; a disease or condition associated with the expression of E2F-dependent genes; a disease or condition associated with proliferation of fibroblast, fibrotic disease or disorder, autoimmune disease or disorder, inflammatory-related disease or disorder, cardiac disease or disorder, cardiovascular disease or disorder, pulmonary disease or disorder, renal disease or disorder, dermal disease or disorder, and any combination thereof. In some embodiments, the fibrotic disease or disorder is selected from the group consisting of renal fibrosis, cardiac fibrosis, cardiovascular fibrosis, pulmonary fibrosis, dermal fibrosis, perivascular myocardial collagen accumulation or deposition, renal collagen accumulation or deposition, dermal collagen accumulation or deposition, pulmonary collagen accumulation or deposition, increased aortic media thickness associated with fibrosis, bronchiolitis olibterans organizing pneumonia (BOOP), acute respiratory distress syndrome (ARDS), asbestosis, accidental radiation induced lung fibrosis, therapeutic radiation induced lung fibrosis, sarcoidosis, silicosis, tuberculosis, COVID-19 associated lung fibrosis, Hermansky Pudlak syndrome, bagassosis, eosinophilic granuloma, Wegener's granulomatosis, lymphangioleiomyomatosis, cystic fibrosis, fatty liver disease, chronic graft-versus-host disease (cGVHD), sclerodermatous graft-versus-host disease, nephrogenic systemic fibrosis, Dupuytren's contracture, keloids, chronic graft rejection, scarring or wound healing abnormalities, post-operative adhesions, reactive fibrosis, disease or disorder associated with nephrotoxic agent exposure, disease or disorder associated with aminoglycoside exposure, disease or disorder associated with non-steroidal anti-inflammatory drug (NSAID) exposure, disease or disorder associated with immune-suppressant exposure, disease or disorder associated with nitrofurantoin exposure, disease or disorder associated with amiodarone exposure, disease or disorder associated with bleomycin exposure, disease or disorder associated with cyclophosphamide exposure, disease or disorder associated with methotrexate exposure, myocardial infarction, injury related tissue scarring, scarring associated with surgery, therapeutic radiation induced fibrosis, dermatomyositis (DM), disease or disorder associated with endothelial cell injury, such as autoimmune-based endothelial cell injury, or any combination thereof.

In some embodiments the disease or condition is a cardiac disease or disorder, such as a cardiac failure, cardiac fibrosis, or any combination thereof.

In some embodiments, the renal disease or disorder is a chronic kidney disease, renal fibrosis, renal disease or disorder associated with systemic lupus erythematosus (SLE), or any combination thereof.

In some embodiments, the compounds disclosed herein are used to treat a pulmonary disease or disorder that is an idiopathic pulmonary fibrosis (IPF) disease, chronic obstructive pulmonary disease (COPD), pulmonary arterial hypertension (PAH), idiopathic pulmonary arterial hypertension (iPAH), pulmonary fibrosis, cystic fibrosis, pulmonary inflammation, or any combination thereof.

In one aspect, the invention provides a method of treating a condition or disease responsive to a modulation of CB₂ activity in a subject. In one embodiment, the method comprises administering to the subject in need thereof a therapeutically effective amount of at least one cannabidiol derivative or composition thereof. In one embodiment, the cannabidiol derivative selectively binds the CB₂. In one embodiment, the R substituent of the compound having the structure of Formula (I) binds the CB₂.

In some embodiments, the condition or disease responsive to the modulation of the CB₂ activity is an autoimmune disease, demyelinating disease, inflammatory-related disorder, fibrotic disease or disorder, cardiac disease or disorder, cardiovascular disease or disorder, or any combination thereof. In some embodiments, the condition or disease responsive to the modulation of the CB₂ activity is a fibrotic disease, cardiac fibrosis, cardiovascular fibrosis, perivascular myocardial collagen deposition, increased aortic media thickness, systemic sclerosis, myelinoclastic disorder, neuromyelitis optica, central nervous system neuropathy, central pontine myelinolysis, myelopathy, leukoencephalopathy, leukodystrophy, peripheral neuropathy, Guillain-Barre syndrome, anti-MAG peripheral neuropathy, Charcot-Marie-Tooth disease, progressive inflammatory neuropathy, or any combination thereof. In one embodiment, the condition or disease responsive to the modulation of the CB₂ activity is cardiac fibrosis.

In another aspect, the invention relates, in part, to a method of treating a condition or disease responsive to the modulation of the E2F transcriptional signature in a subject in need thereof. In one embodiment, the method comprises administering to the subject in need thereof a therapeutically effective amount of at least one cannabidiol derivative or composition thereof. In one embodiment, the cannabidiol derivative modulates the E2F transcriptional signature. In one embodiment, the cannabidiol derivative inhibits the E2F transcriptional signature. In one embodiment, the treated condition or disease is further responsive to the modulation of the CB₂ activity. In one embodiment, the condition or disease responsive to the modulation of the E2F transcriptional signature is further responsive to the modulation of the CB₂ activity.

In some embodiments, the condition or disease responsive to the modulation of the E2F transcriptional signature is an autoimmune disease, demyelinating disease, inflammatory-related disorder, myelinoclastic disorder, neuromyelitis optica, central nervous system neuropathy, central pontine myelinolysis, myelopathy, leukoencephalopathy, leukodystrophy, peripheral neuropathy, Guillain-Barre syndrome, anti-MAG peripheral neuropathy, Charcot-Marie-Tooth disease, progressive inflammatory neuropathy, or any combination thereof.

In another aspect, the invention relates, in part, to compositions comprising a lipidic formulation of the compounds of having the structures of Formula (I)-(X). In some embodiments, the composition comprises a pharmaceutical vehicle that comprises a solubilized of a compound having the structure of Formula (I)-(X).

In one embodiment, the pharmaceutical vehicle is aqueous buffers, solvents, co-solvents, cyclodextrin complexes, lipid vehicles, or any combination thereof, and optionally further comprises at least one stabilizer, emulsifier, polymer, antioxidants, or any combination thereof.

In some embodiments, the lipid vehicle is selected from Captex 300, Tween 85, Cremophor EL, Maisine 35-1, Maisine CC, Capmul MCM, corn oil, and any combination thereof. In some embodiments, the composition includes at least one cannabidiol derivative of the disclosure solubilized in an oil. In other embodiments, the composition comprises at least one cannabidiol derivative of the invention that is solubilized in an oil mixture comprising at least two oils. In one embodiment, the composition comprising at least one cannabidiol derivative of the invention, is solubilized in a Maisine CC: maize oil mixture.

In some embodiments, the lipid vehicle is an oil mixture comprising at least two oils. In some embodiments, the oil mixture is Maisine CC and Maize oil. In some embodiments the Maisine CC and maize oil comprises 50 Maisine CC:50 mainze oil v/v.

In some embodiments of the methods disclosed herein, the compound having the structure of Formula (I)-(X) or a composition thereof is administered in combination with another therapeutic agent. In some embodiments, the compound having the structure of Formula (I)-(X) or a composition thereof is administered before, at the same time, or subsequently with another therapeutic agent.

In some embodiments, the compound having the structure of Formula (I)-(X) or a composition thereof is administered orally, topically, intramuscularly, intravenously, or any combination thereof.

In some embodiments, the compound having the structure of Formula (I)-(X) or a composition thereof is administered with food or drink.

In some embodiments, disclosed herein are methods for preventing or treating a fibrotic disease or disorder in a subject in need thereof, wherein the method involves administering a therapeutically amount of a compound having the structure of Formula (VIII) or a composition thereof:

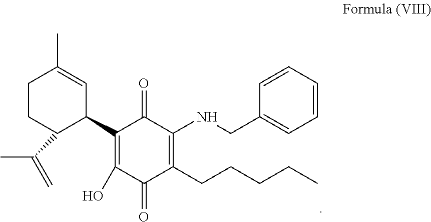

In some embodiments, the fibrotic disease or condition is cardiac fibrosis, aortic fibrosis, pulmonary fibrosis, dermal fibrosis, renal fibrosis, or any combination thereof.

In some embodiments, disclosed herein is a composition for preventing and/or treating a fibrotic disease in a subject in need thereof, wherein the composition comprises a compound having the structure of Formula (I) or a derivative thereof.

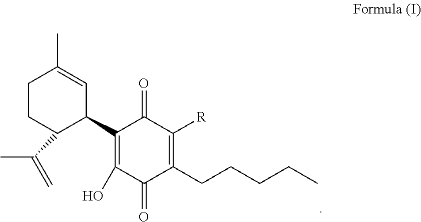

In some embodiments, R is the nitrogen atom of a group independently selected from a linear or branched alkylamine, an arylamine, an arylalkylamine, a heteroarylamine, a heteroarylalkylamine, a linear or branched alkenylamine, a linear or branched alkynylamine, or NH₂.

In some embodiments, the compound having the structure of Formula (I) modulates at least one selected from the group consisting of the activity of E2F pathway, level or activity of at least one E2F-dependent gene, level of myofibroblast, level of fibroblast, level of fibrosis, level of macrophage accumulation, level of macrophage infiltration, level of collagen accumulation or deposition, aortic media thickness, aortic media thickness associated with fibrosis, level or activity of inflammatory response, level or activity of at least one fibrotic protein, level or activity of interferon response, level or activity of IFN-α response, level or activity of IFN-γ response, immune response associated with fibrosis, autoimmune response associated with fibrosis, inflammation associated with fibrosis, or any combination thereof.

In some embodiments, the compound is independently selected from a compound having the structure of:

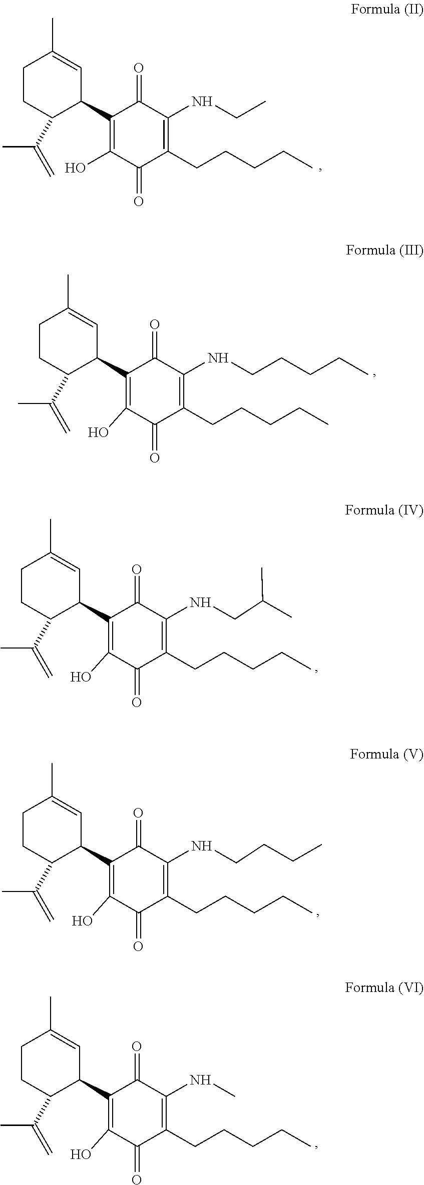

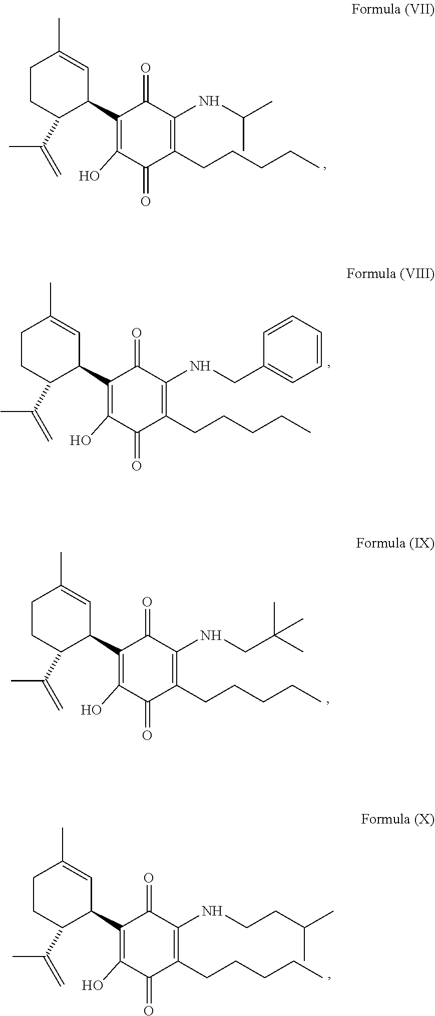

or any combination thereof.

In some embodiments, disclosed herein is a composition comprising at least one compound having the structure of Formula (II)-(X).

### BRIEF DESCRIPTION OF THE DRAWINGS

The following detailed description of various embodiments of the disclosure will be better understood when read in conjunction with the appended drawings. For the purpose of illustrating the disclosure, there are shown in the drawings illustrative embodiments. It should be understood, however, that the disclosure is not limited to the precise arrangements and instrumentalities of the embodiments shown in the drawings.

FIG. 1, comprising FIG. 1A and FIG. 1B, depicts representative results demonstrating the myocardia antifibrotic activity of a Cannabidiol aminoquinone compound having the structure of Formula (VIII) in vivo. Wild-type mice eight weeks of age were anesthetized using isoflurane and an osmotic pump (2004, Alzet, Cupertino, Calif.) delivering Angiotensin II (Ang II) (A9525, Sigma, Madrid, Spain) at 1.0 mg kg⁻¹ min⁻¹, or saline was implanted beneath the midscapular loose skin. Pumps were left for 14 days, one day before the pump implantation, the compound having the structure of Formula (VIII) (20 mg/kg) was administrated by oral gavage (preventive model). Mice were euthanized for tissue collection. FIG. 1A depicts representative Fast green/Sirius red-stained slides of hearts from mice infused with saline, or infused with Ang II and treated with the compound having the structure of Formula (VIII). FIG. 1B depicts representative results for quantification of red-stained collagen on Fast green/Sirius red by ImageJ. Data are expressed as median±interquartile range with statistical comparison between Ang II-treated wild-type mice and all other conditions. For all these area measurements, three to four sections from five to seven mice per group were analyzed. ***p<0.001 versus control group; ###p<0.001 versus Ang II group.

FIG. 2, comprising FIG. 2A and FIG. 2B, depicts representative results demonstrating the effects of the compound having the structure of Formula (VIII) on perivascular myocardial collagen deposition. FIG. 2A depicts representative results demonstrating that the compound having the structure of Formula (VIII) reduced Ang II-induced perivascular myocardial collagen deposition as analyzed by Fast green/Sirius red-stained. FIG. 2B depicts representative results for quantification of respective measurements of perivascular collagen accumulation in left ventricle. Data are expressed as median±interquartile range with statistical comparison between Ang II-treated wild-type mice and all other conditions. For these measurements, all vessels were analyzed from five to seven mice per group were analyzed. ***p<0.001 versus control group; ###p<0.001 versus Ang II group.

FIG. 3 depicts representative results demonstrating the effect of the compound having the structure of Formula (VIII) on cardiac tissue macrophage infiltration in vivo. Mice were infused with Ang II for two weeks and treated in parallel with the oral compound having the structure of Formula (VIII) (20 mg/kg). F4/80+ macrophages in the myocardial tissue were detected by immunostaining, quantified and expressed as F4/80+ cells/area. Data are expressed as median±interquartile range with statistical comparison between Ang II-treated wild-type mice and all other conditions. For all these area measurements, three to four sections from five to seven mice per group were analyzed. ***p<0.001 versus control group; ###p<0.001 versus Ang II group.

FIG. 4, comprising FIG. 4A and FIG. 4B, depicts representative results demonstrating the effect of the compound having the structure of Formula (VIII) on ECM protein Tenascin C (TNC) in cardiac tissue in vivo. Mice were infused with Ang II for two weeks and treated in parallel with the oral compound having the structure of Formula (VIII) (20 mg/kg) beginning one day before Ang II infusion. FIG. 4A depicts representative results demonstrating that TNC in left ventricle sections of heart were detected by immunostaining. FIG. 4B depicts representative results quantified and expressed as TNC percentage of area/image. Results are expressed as median±interquartile range with statistical comparison between Ang II-treated wild-type mice and all other conditions. For all these area measurements, three to four sections five to seven mice per group were analyzed. ***p<0.001 versus control group; ###p<0.001 versus Ang II group.

FIG. 5, comprising FIG. 5A and FIG. 5B, depicts representative results demonstrating aortic antifibrotic activity of the compound having the structure of Formula (VIII) in vivo. Mice were infused with Ang II for two weeks and treated in parallel with the oral compound having the structure of Formula (VIII). FIG. 5A depicts representative sections that were stained with Sirius red to determine the collagen content in adventitial layer. The adventitia area was defined as the area between the external elastic lamina and the tunica externa, the outermost layer of the vessel. FIG. 5B depicts representative results for quantification of respective measurements on aorta sections. For all these area measurements, three to four sections from three to five mice per group were analyzed. Results are expressed as median±interquartile range with statistical comparison between Ang II-treated wild-type mice and all other conditions ***p<0.001 versus control group; ###p<0.001 versus Ang II group.

FIG. 6 depicts representative results demonstrating transcriptomic changes in Ang II-challenged mice treated with the compound having the structure of Formula (VIII). Hallmarks significantly enriched (adjusted p<0.01) in the Ang II vs Control pre-ranked list and in at least one of the other two lists. The direction of the triangles indicates if the enrichment was found in the up or down regulated extreme of the list. The size of the triangles is directly proportional to the significance of the enrichment, as the −log 10 transformed adjusted p value. The analysis was performance with cardiac RNA, n=3 each group.

FIG. 7 depicts representative results demonstrating that compound having the structure of Formula (VIII) reduced the expression on inflammatory and fibrotic marker mRNAs in heart. Gene expression of inflammatory and fibrosis markers including Interleukin 6 (IL6), Interleukin 1 β (IL1β), and TNC was down regulated by the treatment with compound in Ang II mice compared to untreated Ang II mice. Expression levels were calculated using the 2^{'ΔΔCt} method. Values are expressed as means±SEM for three animals per group. #p<0.05 versus Ang II group.

FIG. 8 depicts representative results demonstrating inhibitory effects of the compound having the structure of Formula (VIII) on E2F pathway in cardiac tissue. Gene expression in the E2F pathways markers including CDK1, topoisomerase 2-alpha (TOP2A) and a protein coding gene, the marker of Proliferation Ki-67 (MKi67) was down regulated by the compound in Ang II mice. Expression levels were calculated using the 2^{-ΔΔCt} method. Values are expressed as means±SEM for at seven animals per group. #p<0.05 versus Ang II group.

FIG. 9 depicts representative results demonstrating that compound having the structure of Formula (VIII) reduced myofibroblast proliferation in left ventricle. Myofibroblast proliferation measured in ventricular section stained with an antibody to Ki67 during two weeks after Ang II infusion. Quantification of number of Ki67-positive cells from 5 to 10 fields per heart. Four animals from each group were analyzed. Data are presented as median±interquartile range with statistical comparison between Ang II-treated wild-type mice and all other conditions. ***p<0.001 versus control group; ###p<0.001 versus Ang II group.

FIG. 10, comprising FIG. 10A and FIG. 10B, depicts representative results demonstrating that the compound having the structure of Formula (VIII) prevented renal fibrosis induced by Ang II. FIG. 10A depicts representative photomicrographs of renal tissue from Ang II mouse model stained with Picrosirius red and treated or not with the compound having the structure of Formula (VIII) (20 mg/kg). Arrows indicate the fibrotic areas with collagen deposition. FIG. 10B depicts representative results for quantification of respective measurements of collagen area in kidney. Data are expressed as median±interquartile range with statistical comparison between Ang II-treated wild-type mice and all other conditions. For all these area measurements, three to four sections from three to five mice per group were analyzed. ***p<0.001 versus control group; ###p<0.001 versus Ang II group.

FIG. 11, comprising FIG. 11A and FIG. 11B, depicts representative results demonstrating that the compound having the structure of Formula (VIII) prevented dermal (skin) fibrosis induces by Ang II. FIG. 11A depicts representative photomicrographs of dermal tissue from Ang II mouse model stained with Picrosirius red and treated or not with the compound having the structure of Formula (VIII) (20 mg/kg). FIG. 11B depicts representative results for quantification of respective measurements of collagen area in skin. Data are expressed as median±interquartile range with statistical comparison between Ang II-treated wild-type mice and all other conditions. For all these area measurements, three to four sections from three to five mice per group were analyzed. ***p<0.001 versus control group; ##p<0.01 versus Ang II group.

FIG. 12, comprising FIG. 12A and FIG. 12B, depicts representative results demonstrating effects of the compound having the structure of Formula (VIII) on perivascular myocardial collagen deposition in therapeutic model of Ang II-induced fibrosis. Wild-type mice eight to ten weeks of age were anesthetized using isoflurane and an osmotic pump (2004, Alzet, Cupertino, Calif.) delivering Ang II (A9525, Sigma, Madrid, Spain) at 500 ng kg⁻ min⁻, or saline was implanted beneath the midscapular loose skin. Pumps were left for 28 days, and the compound having the structure of Formula (VIII) (20 mg/kg) were administrated by oral gavage during the last 14 days of the pump implantations (therapeutic model). Mice were euthanized for tissue collection. FIG. 12A depicts representative results demonstrating that the compound having the structure of Formula (VIII) reduced Ang II perivascular myocardial collagen deposition as analyzed by Fast green/Sirius red-staining. FIG. 12B depicts representative results for quantification of respective measurements of perivascular collagen accumulation in left ventricle. Data are expressed as median±interquartile range with statistical comparison between Ang II-treated wild-type mice and all other conditions. For these measurements, all vessels were analyzed from six to thirteen mice per group were analyzed ***p<0.001 versus control group; ##p<0.01 versus Ang II group.

FIG. 13, comprising FIG. 13A and FIG. 13B, depicts representative results demonstrating aorta antifibrotic activity of the compound having the structure of Formula (VIII) in vivo. Mice were infused with Ang II (500 ng kg⁻ min⁻) for four weeks and treated last two weeks with the oral compound having the structure of Formula (VIII) (20 mg/kg). FIG. 13A depicts representative sections that were also stained with Sirius red to determine the collagen content in adventitial layer. FIG. 13B depicts representative data that are expressed as median±interquartile range with statistical comparison between Ang II-treated wild-type mice and all other conditions. For all these area measurements, three to four sections from a sample from six to thirteen mice per group were analyzed. ***p<0.001 versus control group; ###p<0.001 versus Ang II group.

FIG. 14, comprising FIG. 14A and FIG. 14B, depicts representative results demonstrating that the compound having the structure of Formula (VIII) reduced renal fibrosis in the therapeutic Ang II mouse model. Mice were infused with Ang II (500 ng kg⁻ min⁻) for four weeks and treated last two weeks with the oral compound having the structure of Formula (VIII) (20 mg/kg). FIG. 14A depicts representative results of histology evaluation of Sirius red staining of interstitial renal tissue. FIG. 14B depicts representative results for quantification of respective measurements of collagen accumulation in kidney. Data are expressed as median±interquartile range with statistical comparison between Ang II-treated wild-type mice and all other conditions. For all these area measurements, three to four sections from a sample from six to thirteen mice per group were analyzed ***p<0.001 versus control group: #p<0.05 versus Ang II group.

FIG. 15, comprising FIG. 15A and FIG. 15B, depicts representative results demonstrating that the compound having the structure of Formula (VIII) reduced lung fibrosis in the therapeutic Ang II mouse model. Mice were infused with Ang II (500 ng kg⁻ min⁻) for four weeks and treated last two weeks with the oral compound having the structure of Formula (VIII) (20 mg/kg). FIG. 15A depicts representative results for histology evaluation of Masson trichrome staining of lungs. FIG. 15B depicts representative results for quantification by Ashcroft score in lung. Ang II-treated wild-type mice and all other conditions. For all these area measurements, three to four sections from a sample from six to thirteen mice per group were analyzed ***p<0.001 versus control group: ###p<0.001 versus Ang II group.

FIG. 16, comprising FIG. 16A through FIG. 16C, depicts representative results demonstrating that compound having the structure of Formula (VIII) (also named VCE-004.8) decreased α-SMA expression in in cultured human immortalized cardiac fibroblasts (hICF) treated with either TGF-β or Ang II and inhibited both Ang II-induced ERK1+2 phosphorylation and NF-AT activation in hICF. FIG. 16A depicts representative results demonstrating that compound having the structure of Formula (VIII) (also named VCE-004.8) decreased α-SMA expression in in cultured human immortalized cardiac fibroblasts (hICF) treated with either TGF-β or Ang II. FIG. 16B depicts representative results demonstrating that compound having the structure of Formula (VIII) (also named VCE-004.8) as well as Losartan inhibited Ang II-induced ERK1+2 phosphorylation. Data represent the mean±SEM, and significance was determined by Kruskall-Wallis followed by Dunn's post-hoc test. **p<0.01 vs control, #p<0.05, and ##p<0.01 vs Ang II. FIG. 16C depicts representative results demonstrating that compound having the structure of Formula (VIII) (also named VCE-004.8) as well as Losartan inhibited NF-AT activation in hICF. Data represent the mean±SEM, and significance was determined by Kruskall-Wallis followed by Dunn's post-hoc test. *p<0.05 vs Ang II, **p<0.01 vs Ang II.

FIG. 17 depicts representative results demonstrating that compound having the structure of Formula (VIII) (also named VCE-004.8) inhibited the mRNA expression of IL1b, IL6, Col1A2 and CCL2, induced by Ang II or TGFb in hICF cells. Data represent the mean±SEM, and significance was determined by Kruskall-Wallis followed by Dunn's post-hoc test. *p<0.05, **p<0.01, and ***p<0.001 vs control; #p<0.05, and ##p<0.01 vs Ang II.

### DETAILED DESCRIPTION

It is to be understood that the Figures and descriptions of the present invention have been simplified to illustrate elements that are relevant for a clear understanding of the present invention, while eliminating, for the purpose of clarity, many other elements found in the method of treating a condition or disease responsive to a modulation of CB₂ activity or condition or disease responsive to the modulation of the E2F transcriptional signature using various cannabidiol derivatives, such as the compounds having the structure of Formula (I), as well as methods of making and using such compounds, pharmaceutical compositions, and liquid formulations thereof. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

The present disclosure relates to compositions comprising one or more cannabidiol derivatives of Formula (I) and the use of these cannabidiol quinone derivatives of Formula (I) in the treatment of various diseases or disorders, such as cardiac, aortic, renal, skin, pulmonary fibrotic disease or disorder, or any combination thereof. In some embodiments, the present disclosure relates to methods of preventing or treating cardiac fibrosis, aortic fibrosis, renal fibrosis, skin, pulmonary fibrosis, or any combination thereof using cannabidiol aminoquinone derivative of Formula (VIII) or compositions thereof.

### Definitions

As used herein, each of the following terms has the meaning associated with it in this section. Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent depending on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

The terms “patient,” “subject,” “individual,” and the like are used interchangeably herein, and refer to any animal, or cells thereof whether in vitro or in situ, amenable to the methods described herein. In certain non-limiting embodiments, the patient, subject, or individual is a mammal, non-human mammal, primate, mouse, rat, pig, horse, ferret, dog, cat, cattle, or human.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a subject and includes: (a) preventing a disease related to an undesired immune response from occurring in a subject which may be predisposed to the disease; (b) inhibiting the disease, i.e., arresting its development: or (c) relieving the disease, i.e., causing regression of the disease.

The term “inhibit,” as used herein, means to suppress or block an activity or function by at least about ten percent relative to a control value. In some embodiments, the activity is suppressed or blocked by 50% compared to a control value. In some embodiments, the activity is suppressed by 75%. In some embodiments, the activity is suppressed by 95%.

In the context of the present disclosure, a “modulator” is defined as a compound that is an agonist, a partial agonist, an inverse agonist or an antagonist (e.g., agonist, a partial agonist, an inverse agonist or an antagonist of E2F). For example, a modulator may increase the activity of the E2F pathway or may decrease the activity of the E2F pathway. Additionally, an “agonist” is defined as a compound that increases the basal activity of a receptor (i.e., signal transduction mediated by the receptor). An “antagonist” is defined as a compound, which blocks the action of an agonist on a receptor. A “partial agonist” is defined as an agonist that displays limited, or less than complete, activity such that it fails to activate a receptor in vitro, functioning as an antagonist in vivo. An “inverse agonist” is defined as a compound that decreases the basal activity of a receptor.

The term “derivative” refers to a small molecule that differs in structure from the reference molecule, but may retain or enhance the essential properties of the reference molecule and may have additional properties. A derivative may change its interaction with certain other molecules relative to the reference molecule. A derivative molecule may also include a salt, an adduct, tautomer, isomer, or other variant of the reference molecule.

The term “tautomers” are constitutional isomers of organic compounds that readily interconvert by a chemical process (tautomerization).

The term “isomers” or “stereoisomers” refers to compounds, which have identical chemical constitution, but differ with regard to the arrangement of the atoms or groups in space.

As used herein “polymorph” refers to crystalline forms having the same chemical composition but different spatial arrangements of the molecules, atoms, and/or ions forming the crystal.

As used herein, “alkyl” refers to a linear or branched chain fully saturated (no double or triple bonds) hydrocarbon (all carbon) group. An alkyl group of this invention may comprise from 1-20 carbon atoms, that is, “m”=1 and “n”=20, designated as a “C₁ to C₂₀ alkyl.” In one embodiment, “m”=1 and “n”=12 (C₁ to C₁₂ alkyl). In other embodiments, that “m”=1 and “n”=6 (C₁ to C₆ alkyl). Examples of alkyl groups include, without limitation, methyl, ethyl, n-propyl, isopropyl, n-butyl, iso-butyl, sec-butyl, tert-butyl, amyl, tert-amyl, hexyl, heptyl, octyl, nonyl, decyl, undecyl, and dodecyl.

An alkyl group of this invention may be substituted or unsubstituted. When substituted, the substituent group(s) is(are) one or more group(s) independently selected from cycloalkyl, aryl, heteroaryl, heteroalicyclyl, hydroxy, alkoxy, aryloxy, mercapto, alkylthio, arylthio, cyano, halo, oxo, carbonyl, thiocarbonyl, O-carbamyl, N-carbamyl, O-thiocarbamyl, N-thiocarbamyl, C-amido, N-amido, S-sulfonamido, N-sulfonamido, C-carboxy, O-carboxy, isocyanato, thiocyanato, isothiocyanato, nitro, silyl, trihalomethanesulfonyl, —NRaRb, protected hydroxyl, protected amino, protected carboxy, and protected amido groups.

Examples of substituted alkyl groups include, without limitation, 2-oxo-prop-1-yl, 3-oxo-but-1-yl, cyanomethyl, nitromethyl, chloromethyl, hydroxymethyl, tetrahydropyranyloxymethyl, m-trityloxymethyl, propionyloxymethyl, aminomethyl, carboxymethyl, allyloxycarbonylmethyl, allyloxycarbonylaminomethyl, methoxymethyl, ethoxymethyl, t-butoxymethyl, acetoxymethyl, chloromethyl, bromomethyl, iodomethyl, trifluoromethyl, 6-hydroxyhexyl, 2,4-dichlorobutyl, 2-aminopropyl, 1-chloroethyl, 2-chloroethyl, 1-bromoethyl, 2-chloroethyl, 1-fluoroethyl, 2-fluoroethyl, 1-iodoethyl, 2-iodoethyl, 1-chloropropyl, 2-chloropropyl, 3-chloropropyl, 1-bromopropyl, 2-bromopropyl, 3-bromopropyl, 1-fluoropropyl, 2-fluoropropyl, 3-fluoropropyl, 1-iodopropyl, 2-iodopropyl, 3-iodopropyl, 2-aminoethyl, 1-aminoethyl, N-benzoyl-2-aminoethyl, Nacetyl-2-aminoethyl, N-benzoyl-1-aminoethyl, and N-acetyl-1-aminoethyl.

As used herein, “alkenyl” refers to an alkyl group that contains in a linear or branched hydrocarbon chain one or more double bonds. Examples of alkenyl groups include, without limitation, vinyl (CH₂═CH—), allyl (CH₃CH═CH₂—), 1-propenyl, 2-propenyl, 1-butenyl, 2-butenyl; 1-pentenyl, 2-pentenyl, 3-pentenyl, 4-pentenyl, 3-methyl-1-butenyl, and the various isomers of hexenyl, heptenyl, octenyl, nonenyl, decenyl, undecenyl, and dodecenyl.

An alkenyl group of this invention may be unsubstituted or substituted. When substituted, the substituent(s) may be selected from the same groups disclosed above with regard to alkyl group substitution. Examples of substituted alkenyl groups include, without limitation, styrenyl, 3-chloro-propen-1-yl, 3-chloro-buten-1-yl, 3-methoxy-propen-2-yl, 3-phenyl-buten-2-yl, and 1-cyano-buten3-yl.

As used herein, “alkynyl” refers to an alkyl group that contains in a linear or branched hydrocarbon chain one or more triple bonds.

An alkynyl group of this invention may be unsubstituted or substituted. When substituted, the substituent(s) may be selected from the same groups disclosed above with regard to alkyl group substitution.

As used herein, “aryl” refers to a carbocyclic (all carbon) ring or two or more fused rings (rings that share two adjacent carbon atoms) that have a fully delocalized pi-electron system. Examples of aryl groups include, but are not limited to, benzene, and substituted benzene, such as toluene, aniline, xylene, and the like, naphthalene and substituted naphthalene, and azulene.

The term “pharmaceutically acceptable salt” refers to any pharmaceutically acceptable salt, which upon administration to the patient is capable of providing (directly or indirectly) a compound as described herein. Such salts preferably are acid addition salts with physiologically acceptable organic or inorganic acids. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methane sulphonate and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic amino acids salts. However, it will be appreciated that non-pharmaceutically acceptable salts also fall within the scope of the invention since those may be useful in the preparation of pharmaceutically acceptable salts. Procedures for salt formation are conventional in the art.

The term “solvate” in accordance with this invention should be understood as meaning any form of the active compound in accordance with the invention in which said compound is bonded by a non-covalent bond to another molecule (normally a polar solvent), including especially hydrates and alcoholates.

The terms “effective amount” and “pharmaceutically effective amount” refer to a sufficient amount of an agent to provide the desired biological result. That result can be reduction and/or alleviation of a sign, symptom, or cause of a disease or disorder, or any other desired alteration of a biological system. An appropriate effective amount in any individual case may be determined by one of ordinary skill in the art using routine experimentation.

A “therapeutically effective amount” refers to that amount which provides a therapeutic effect for a given condition and administration regimen. In particular, “therapeutically effective amount” means an amount that is effective to prevent, alleviate or ameliorate symptoms of the disease or prolong the survival of the subject being treated, which may be a human or non-human animal. Determination of a therapeutically effective amount is within the skill of the person skilled in the art.

As used herein, the term “pharmaceutical composition” refers to a mixture of at least one compound of the invention with other chemical components and entities, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

Various aspects of the disclosure can be presented in a range format. It should be understood that the description in range format is merely for convenience and brevity and should not be construed as an inflexible limitation on the scope of the disclosure.

### Description

The present disclosure relates to compositions, comprising the cannabidiol derivatives of Formula (I) as well as the use of these cannabidiol aminoquinone derivatives or compositions thereof in the treatment of various diseases or disorders, such as cardiac, renal, aortic, skin, or pulmonary fibrotic disease or disorder. In some embodiments, the present disclosure relates to methods of preventing or treating cardiac, renal, aortic, skin, or pulmonary fibrotic disease or disorder using cannabidiol aminoquinone derivatives of Formula (II)-(X) or compositions thereof. In some embodiments, the present disclosure relates to methods of preventing or treating cardiac, renal, aortic, skin, or pulmonary fibrotic disease or disorder using cannabidiol aminoquinone derivatives of Formula (VIII) or compositions thereof.

### Compositions

The disclosure provides a cannabidiol aminoquinone derivatives that modulate the activity of E2F pathway, level or activity of at least one E2F-dependent gene, level of fibrosis, level of fibroblast, level of myofibroblast, level of macrophage accumulation, level of macrophage infiltration, level of collagen accumulation or deposition, aortic media thickness, aortic media thickness associated with fibrosis, level or activity of inflammatory response, level or activity of at least one fibrotic protein, level or activity of interferon response, level or activity of interferon α (IFN-α) response, level or activity of interferon γ (IFN-γ) response, immune response associated with fibrosis, autoimmune response associated with fibrosis, inflammation associated with fibrosis, or any combination thereof.

In one embodiment, the cannabidiol derivative is a synthetic cannabidiol derivative. In one embodiment, the synthetic cannabidiol derivative comprises chemically stable, nonpsychotropic aminoquinone chemically derived from synthetic cannabidiol through oxidation and amination. In one embodiment, the synthetic cannabidiol derivative comprises chemically stable, nonpsychotropic aminoquinone chemically derived from natural cannabidiol through oxidation and amination. In one embodiment, the synthetic cannabidiol derivative is a non-reactive synthetic cannabidiol aminoquinone derivative. In one embodiment, the non-reactive synthetic cannabidiol derivative is a chemically stable synthetic cannabidiol derivative. In one embodiment, the non-reactive synthetic cannabidiol derivative is a synthetic cannabidiol aminoquinone derivative that does not have a detectable affinity for the CB1 receptor.

In one embodiment, the cannabidiol derivative is a compound having the structure of Formula (I).

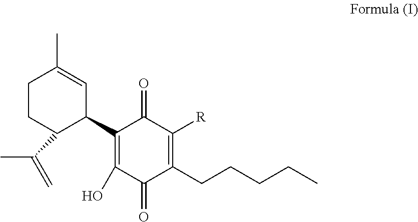

In one embodiment, R is the nitrogen atom of a group independently selected from a linear or branched alkylamine, an arylamine, an arylalkylamine, a heteroarylamine, a heteroarylalkylamine, a linear or branched alkenylamine, a linear or branched alkynylamine, or NH₂.

In one embodiment, the cannabidiol derivative is a cannabidiol aminoquinone derivative. For example, in some embodiments, the cannabidiol aminoquinone derivative is selected from:

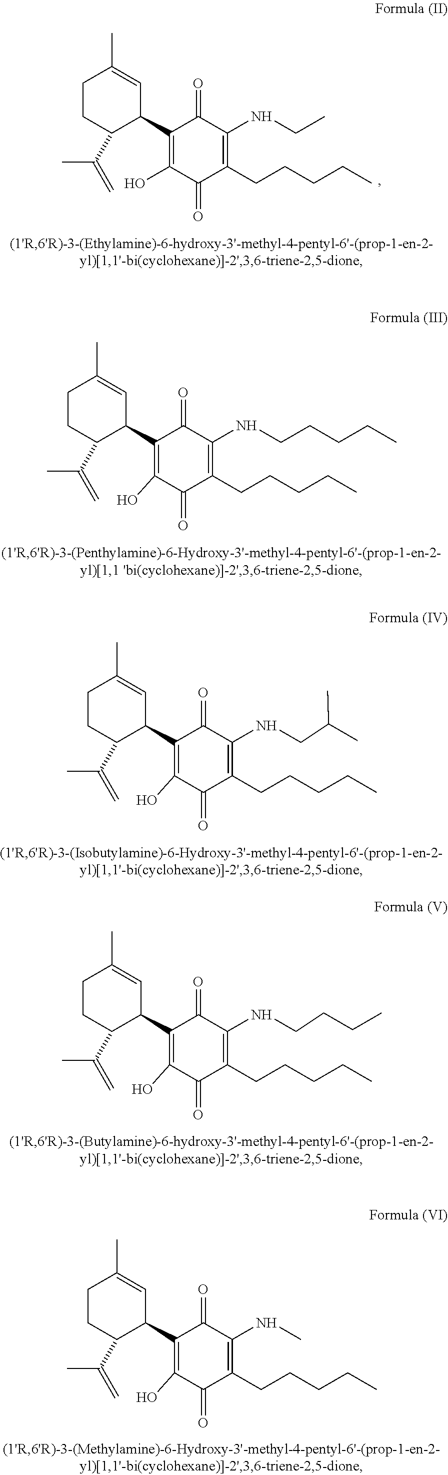

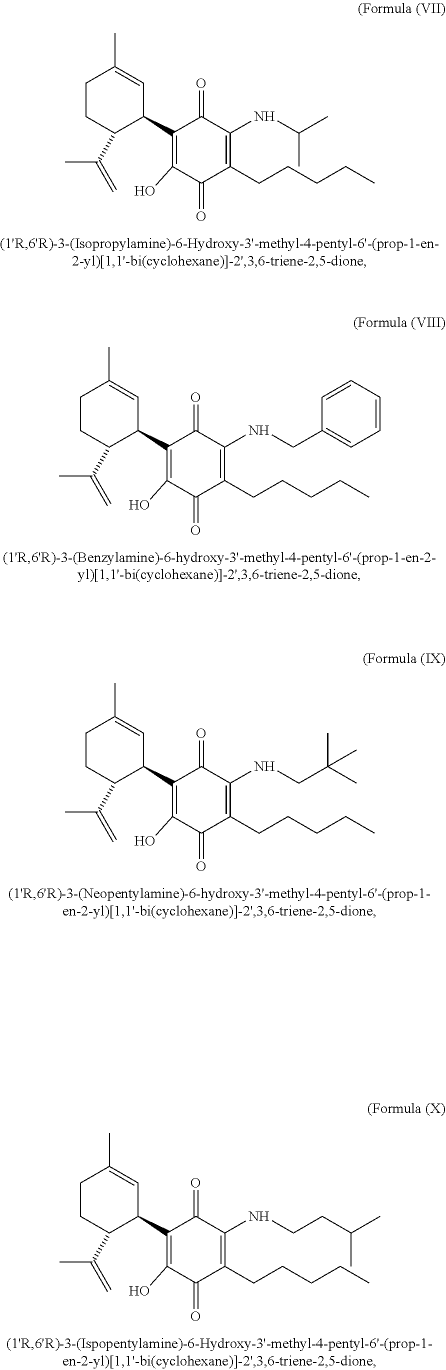

or any combination thereof.

In some embodiments, the cannabidiol aminoquinone derivative is, optionally, a modulator of PPARγ, CB₂ receptor signaling, stabilizes HIF, or any combination thereof. Thus, in some embodiments, the cannabidiol aminoquinone derivative is, optionally, a modulator of PPARγ, CB₂ receptor signaling, E2F activity (e.g., level, activity, expression, etc.), stabilizes HIF, or any combination thereof. In one embodiment, the cannabidiol aminoquinone derivative is a modulator of PPARγ, CB₂ receptor signaling, and E2F activity (e.g., level, activity, expression, etc.), and stabilizes HIF-1α thus upregulating the expression of several associated factors that include Erythropoietin (EPO) and Vascular Endothelial Growth Factor A (VEGFA). For example, in one embodiment, the cannabidiol aminoquinone derivative reduces fibrosis by acting on PPARγ, CB₂ receptors, E2F activity (e.g., level, activity, expression, etc.), HIF pathway, or any combination thereof.

In one aspect, the disclosure also provides a composition comprising at least one cannabidiol aminoquinone derivative solubilized in a pharmaceutical vehicle. In one embodiment, the composition comprises new drug candidates comprising chemically stable, nonpsychotropic aminoquinone chemically derived from synthetic or natural cannabidiol through oxidation and amination.

In various embodiments, the composition modulates the activity of E2F pathway, level or activity of at least one E2F-dependent gene, level of fibrosis, level of fibroblast, level of myofibroblast, level of macrophage accumulation, level of macrophage infiltration, level of collagen accumulation or deposition, aortic media thickness, aortic media thickness associated with fibrosis, level or activity of inflammatory response, level or activity of at least one fibrotic protein, level or activity of interferon response, level or activity of IFN-α response, level or activity of IFN-γ response, immune response associated with fibrosis, autoimmune response associated with fibrosis, inflammation associated with fibrosis, or any combination thereof.

In one embodiment, the composition has increased bioavailability. In one embodiment, the composition has increased bioavailability when compared to the bioavailability of the same cannabidiol aminoquinone derivative in a non-formulated mixture. In one embodiment, the composition has increased solubility. In one embodiment, the composition has improved solubility when compared to the solubility of the same cannabidiol aminoquinone derivative in a non-formulated mixture. For example, in some embodiments, the cannabidiol aminoquinone derivative or composition thereof solubilized in a pharmaceutical vehicle has a solubility range of about 0.001 mg/mL-about 10.0 g/mL.

In one embodiment, the pharmaceutical vehicle is selected from the group consisting of aqueous buffers, solvents, co-solvents, cyclodextrin complexes, lipid vehicles, and any combination thereof, and optionally further comprising at least one stabilizer, emulsifier, polymer, antioxidant, and any combination thereof.

In one embodiment, the solvent is selected from the group consisting of acetone, ethyl acetate, acetonitrile, pentane, hexane, heptane, methanol, ethanol, isopropyl alcohol, dimethyl sulfoxide (DMSO), water, chloroform, dichloromethane, diethyl ether, PEG400, Transcutol (diethylene glycomonoethyl ether), MCT 70, Labrasol (PEG-8 caprylic/capric glycerides), Labrafil M1944CS (PEG 5 Oleate), propylene glycol, Transcutol P, PEG400, propylene glycol, glycerol, Captex 300, Tween 85, Cremophor EL, Maisine 35-1, Maisine CC, Capmul MCM, maize oil (also referred to as corn oil), and any combination thereof.

In one embodiment, the co-solvent is selected from the group consisting of acetone, ethyl acetate, acetonitrile, pentane, hexane, heptane, methanol, ethanol, isopropyl alcohol, DMSO, water, chloroform, dichloromethane, diethyl ether, PEG400, Transcutol (diethylene glycomonoethyl ether), MCT 70, Labrasol (PEG-8 caprylic/capric glycerides), Labrafil M1944CS (PEG 5 Oleate), propylene glycol, Transcutol P, PEG400, propylene glycol, glycerol, Captex 300, Tween 85, Cremophor EL, Maisine 35-1, Maisine CC, Capmul MCM, maize oil (also referred to as corn oil), and any combination thereof.

In one embodiment, the cyclodextrin complexes is selected from the group consisting of methyl-β-cyclodextrin, methyl-γ-cyclodextrin, HP-β-cyclodextrin, HP-γ-cyclodextrin, SBE-β-cyclodextrin, α-cyclodextrin, γ-cyclodextrin,6-O-glucosyl-β-cyclodextrin, and any combination thereof.

In one embodiment, the lipid vehicle is selected from the group consisting of Captex® 300, Tween® 85, Cremophor EL, Maisine® 35-1, Maisine CC, Capmul® MCM, maize oil (also referred to as corn oil), and any combination thereof. In one embodiment, the lipid vehicle is an oil. In one embodiment, the lipid vehicle is an oil mixture. In one embodiment, the oil mixture comprises at least two oils. In one embodiment, the oil is selected from the group consisting of Captex 300, Tween 85, Cremophor EL, Maisine 35-1, Maisine CC, Capmul MCM, maize oil (also referred to as corn oil), and any combination thereof. For example, in some embodiments, the oil mixture is from about 10:90 v/v oil mixture to about 90:10 v/v oil mixture.

In one embodiment, the oil mixture is 10:90 v/v oil mixture. In one embodiment, the oil mixture is 20:80 v/v mixture. In one embodiment, the oil mixture is 30:70 v/v oil mixture. In one embodiment, the oil mixture is 40:60 v/v oil mixture. In one embodiment, the oil mixture is 42:58 v/v oil mixture. In one embodiment, the oil mixture is 50:50 v/v oil mixture. In one embodiment, the oil mixture is 55:45 v/v oil mixture. In one embodiment, the oil mixture is 60:40 v/v oil mixture. In one embodiment, the oil mixture is 70:30 v/v oil mixture. In one embodiment, the oil mixture is 80:20 v/v oil mixture. In one embodiment, the oil mixture is 90:10 v/v oil mixture.

In one embodiment, the stabilizer is selected from the group consisting of Pharmacoat® 603, SLS, Nisso HPC-SSL, Kolliphor®, PVP K30, PVP VA 64, and any combination thereof. In one embodiment, the stabilizer is an aqueous solution.

In one embodiment, the polymer is selected from the group consisting of HPMC-AS-MG, HPMC-AS-LG, HPMC-AS-HG, HPMC, HPMC-P-55S, HPMC-P-50, methyl cellulose, HEC, HPC, Eudragit L100, Eudragit® E100, PEO 100K, PEG 6000, PVP VA64, PVP K30, TPGS, Kollicoat® IR, Carbopol® 980NF, Povocoat MP, Soluplus®, Sureteric®, Pluronic® F-68, and any combination thereof.

In one embodiment, the antioxidant is selected from the group consisting of Vitamin A, Vitamin C, Vitamin E, Coenzyme Q10, manganese, iodide, melatonin, alpha-carotene, astaxanthin, beta-carotene, canthaxanthin, cryptoxanthin, lutein, lycopene, zeaxanthin, polyphenol antioxidant, flavonoid, flavones, apigenin, luteolin, tangeritin, flavonol, isorhammetin, kaempferol, myricetin, proanthocyanidin, quercetin, flavanone, eriodictyol, hesperetin, naringenin, flavanol, catechin, gallocatechin, gallate esters, epicatechin, epigallocatechin, theaflavin, thearubigin, isoflavone phytoestrogen, daidzein, genistein, glycitein, stilbenoid, resveratrol, pterostilbene, anthocyanin, cyanidin, delphinidin, malvidin, pelargonidin, peonidin, petunidin, chicoric acid, caffeic acid, chlorogenic acid, ferulic acid, cinnamic acid, ellagic acid, ellagitannin, gallic acid, gallotannin, rosmarinic acid, salicylic acid, curcumin, flavonolignan, silymarin, xanthone, eugenol, capsaicin, bilirubin, citric acid, oxalic acid, phytic acid, n-acetylcysteine, R-alpha-lipoic acid, and any combination thereof.

In one embodiment, the aqueous buffer is selected from the group consisting of aqueous HCl, aqueous citrate-HCl buffer, aqueous NaOH, aqueous citrate-NaOH buffer, aqueous phosphate buffer, aqueous KCl, aqueous borate-KCl-NaOH buffer, PBS buffer, and any combination thereof. For example, in some embodiments, the aqueous buffer has pH range of about pH=0.5-10. In some embodiments, the aqueous buffer has a concentration range of about 0.05 N-about 1.0 N.

In one embodiment, the cannabidiol derivative or composition thereof solubilized in a pharmaceutical vehicle has a solubility range of 0.001 mg/mL-10.0 g/mL. In one embodiment, the cannabidiol derivative or composition thereof has a solubility of 0.001 mg/mL. In one embodiment, the cannabidiol derivative or composition thereof has a solubility of 0.005 mg/mL. In one embodiment, the cannabidiol derivative or composition thereof has a solubility of 0.006 mg/mL. In one embodiment, the cannabidiol derivative or composition thereof has a solubility of 0.008 mg/mL. In one embodiment, the cannabidiol derivative or composition thereof has a solubility of 0.01 mg/mL. In one embodiment, the cannabidiol derivative or composition thereof has a solubility of 0.03 mg/mL. In one embodiment, the cannabidiol derivative or composition thereof has a solubility of 0.06 mg/mL. In one embodiment, the cannabidiol derivative or composition thereof has a solubility of 1.0 mg/mL. In one embodiment, the cannabidiol derivative or composition thereof has a solubility of 2.0 mg/mL. In one embodiment, the cannabidiol derivative or composition thereof has a solubility of 2.5 mg/mL. In one embodiment, the cannabidiol derivative or composition thereof has a solubility of 6.1 mg/mL. In one embodiment, the cannabidiol derivative or composition thereof has a solubility of 10.0 mg/mL. In one embodiment, the cannabidiol derivative or composition thereof has a solubility of 10.2 mg/mL. In one embodiment, the cannabidiol derivative or composition thereof has a solubility of 100.0 mg/mL. In one embodiment, the cannabidiol derivative or composition thereof has a solubility of 250.0 mg/mL. In one embodiment, the cannabidiol derivative or composition thereof has a solubility of 500.0 mg/mL. In one embodiment, the cannabidiol derivative or composition thereof has a solubility of 750.0 mg/mL. In one embodiment, the cannabidiol derivative or composition thereof has a solubility of 1.0 g/mL. In one embodiment, the cannabidiol derivative or composition thereof has a solubility of 1.5 g/mL. In one embodiment, the cannabidiol derivative or composition thereof has a solubility of 5.0 g/mL. In one embodiment, the cannabidiol derivative or composition thereof has a solubility of 8.0 g/mL. In one embodiment, the cannabidiol derivative or composition thereof has a solubility of 10.0 g/mL.

For example, in one embodiment, the cannabidiol derivative of the present invention is formulated in a mixture of Maisine CC:maize oil (50:50 v/v). In one embodiment, the cannabidiol derivative of the present invention is formulated in a mixture of Maisine CC:maize oil (50:50 w/w). Thus, in one embodiment, the composition of the present invention comprises about 20 mg cannabidiol derivative of the present invention, about 490 mg Maisine CC, and about 490 mg maize oil.

In one embodiment, the cannabidiol derivative of the present invention is formulated in a mixture of Maisine 35-1:maize oil (50:50 v/v). In one embodiment, the cannabidiol derivative of the present invention is formulated in a mixture of Maisine 35-1: maize oil (50:50 w/w). Thus, in one embodiment, the composition of the present invention comprises about 20 mg cannabidiol derivative of the present invention, about 490 mg Maisine 35-1, and about 490 mg maize oil.

In various embodiments, the composition of the present invention comprises any composition disclosed in PCT Application No.: PCT/US2020/017035, disclosure of which is incorporated herein by reference in its entirety.

In some embodiments, the cannabidiol aminoquinone derivative or composition thereof modulates the activity of E2F pathway, level or activity of at least one E2F-dependent gene, level of myofibroblast, level of fibroblast, level of fibrosis, level of macrophage accumulation, level of macrophage infiltration, level of collagen accumulation or deposition, aortic media thickness, aortic media thickness associated with fibrosis, level or activity of inflammatory response, level or activity of at least one fibrotic protein, level or activity of interferon response, level or activity of IFN-α response, level or activity of IFN-γ response, immune response associated with fibrosis, autoimmune response associated with fibrosis, inflammation associated with fibrosis, or any combination thereof.

Examples of collagen accumulations or depositions include, but are not limited to interstitial collagen accumulation or deposition, interstitial collagen accumulation or deposition in cardiac tissue, myocardial collagen accumulation or deposition, perivascular myocardial collagen accumulation or deposition, renal collagen accumulation or deposition, dermal (skin) collagen accumulation or deposition, pulmonary collagen accumulation or deposition, or any combination thereof.

In various embodiments, the cannabidiol aminoquinone derivative or composition thereof modulates the level of fibrosis in any fibrotic condition, disease, or disorder regardless of whether the fibrosis is the result of disease, accidental exposure to radiation, accidental tissue injury, therapeutic exposure to radiation, or surgical procedures. Thus, it is understood that the disclosed cannabidiol aminoquinone derivative or composition thereof modulates the level of fibrosis in fibrotic diseases wherein the cause of the fibrosis includes, but is not limited to pulmonary fibrosis, idiopathic pulmonary fibrosis (IPF), bronchiolitis obliterans organizing pneumonia (BOOP), dermatomyositis (DM), acute respiratory distress syndrome (ARDS), asbestosis, accidental radiation induced lung fibrosis, therapeutic radiation induced lung fibrosis, sarcoidosis, silicosis, tuberculosis, COVID-19 associated lung fibrosis, Hermansky Pudlak syndrome, bagassosis, systemic lupus erythematosus (SLE), eosinophilic granuloma, Wegener's granulomatosis, lymphangioleiomyomatosis, cystic fibrosis, fatty liver disease, chronic graft-versus-host disease (cGVHD), sclerodermatous graft versus host disease, Nephrogenic Systemic Fibrosis ((NSF also referred to as nephrogenic fibrosing dermopathy (NFD)), nephrotoxic agent exposure, aminoglycoside exposure, non-steroidal anti-inflammatory drug (NSAID) exposure, immune-suppressant exposure, nitrofurantoin exposure, amiodarone exposure, bleomycin exposure, cyclophosphamide exposure, methotrexate exposure, endothelial cell injury, such as autoimmune-based endothelial cell injury, myocardial infarction, injury related tissue scarring, scarring form surgery, therapeutic radiation induced fibrosis, or any combination thereof.

In some embodiments, the cannabidiol aminoquinone derivative or composition thereof reduces the activity of E2F pathway, level or activity of at least one E2F-dependent gene, level of myofibroblast, level of fibroblast, level of fibrosis, level of macrophage accumulation, level of macrophage infiltration, level of collagen accumulation or deposition, aortic media thickness, aortic media thickness associated with fibrosis, level or activity of inflammatory response, level or activity of at least one fibrotic protein, level or activity of interferon response, level or activity of IFN-α response, level or activity of IFN-γ response, immune response associated with fibrosis, autoimmune response associated with fibrosis, inflammation associated with fibrosis, or any combination thereof.

In other embodiments, the cannabidiol aminoquinone derivative or composition thereof inhibits the activity of E2F pathway, activity of at least one E2F-dependent gene, proliferation of myofibroblast, proliferation of fibroblast, proliferation of fibrosis, macrophage accumulation, macrophage infiltration, collagen accumulation or deposition, development of aortic media thickness, development of aortic media thickness associated with fibrosis, activity of inflammatory response, activity of at least one fibrotic protein, activity of interferon response, activity of IFN-α response, activity of IFN-γ response, immune response associated with fibrosis, autoimmune response associated with fibrosis, inflammation associated with fibrosis, and any combination thereof.

In one embodiment, the cannabidiol aminoquinone derivative or composition thereof modulates the level (e.g., expression, activity, level, etc.) of at least one E2F-dependent gene. Examples of E2F-dependent gene include, but are not limited to CDK1 gene, TOP2A gene, and MKi67 gene.

In some embodiments, the cannabidiol aminoquinone derivative or composition thereof further modulates the level (e.g., expression, activity, level, etc.) of IL6 gene, IL1β gene, TNC gene, at least one E2F-dependent gene, at least one inflammatory gene, at least one profibrotic gene, or any combination thereof. In some embodiments, the cannabidiol aminoquinone derivative or composition thereof further reduces the level (e.g., expression, activity, level, etc.) of IL6 gene, IL1β gene, TNC gene, at least one E2F-dependent gene, at least one inflammatory gene, at least one profibrotic gene, or any combination thereof. In some embodiments the cannabidiol aminoquinone derivative or composition thereof further inhibits the level (e.g., expression, activity, level, etc.) of IL6 gene, IL1β gene, TNC gene, at least one E2F-dependent gene, at least one inflammatory gene, at least one profibrotic gene, or any combination thereof.

In some embodiments, the cannabidiol aminoquinone derivative or composition thereof decreased α-SMA expression in cultured human immortalized cardiac fibroblasts (hICF) treated with either TGF-β or Ang II, and inhibited both Ang II-induced ERK1+2 phosphorylation and NF-AT activation in hICF.

In some embodiments, the cannabidiol aminoquinone derivative or composition thereof inhibited the mRNA expression of IL1b, IL6, Col1A2 and CCL2, induced by Ang II or TGF-β in hICF cells.

In some embodiments, the compound of Formula (I)-(X), optionally, comprises E2F regulating properties, anti-inflammatory properties, or a combination thereof. Thus, the compositions comprising at least one compound of Formula (I)-(X) are useful in treating or preventing a disease or condition associated with E2F pathway, disease or condition associated with inflammation, or any combination thereof.

Furthermore, in various embodiments, the compounds of Formula (I)-(X) or a composition thereof are useful in treating or preventing a disease or condition associated with E2F pathway, disease or condition associated with inflammation, such as systemic inflammation or pulmonary inflammation, disease or condition associated with fibroblast, such as renal fibroblast, disease or condition associated with proliferation of fibroblast, disease or condition associated with myofibroblast, such as renal myofibroblast or dermal myofibroblast, disease or condition associated with proliferation of myofibroblast, disease or condition associated with collagen, disease or condition associated with collagen accumulation or deposition, disease or condition associated with aortic media thickness, disease or condition associated with increased aortic media thickness, disease or condition associated with increased aortic media thickness by fibrosis, disease or condition associated with macrophage infiltration, disease or condition associated with fibrotic proteins, disease or condition associated with fibrotic proteins expression, disease or condition associated with E2F-dependent genes, disease or condition associated with the expression of E2F-dependent genes, fibrotic disease or disorder, autoimmune disease or disorder, such as SLE, cystic fibrosis, inflammatory-related disease or disorder, cardiac disease or disorder, cardiovascular disease or disorder, renal disease or disorder, dermal (skin) disease or disorder, pulmonary disease or disorder (also referred to as lung disease or disorder), disease or disorder associated with autoimmune disease or disorder, such as a disease or disorder associated with SLE, or any combination thereof.

Examples of fibrotic diseases or disorders include, but are not limited to, renal fibrosis, cardiac fibrosis, cardiovascular fibrosis, aortic fibrosis, dermal (skin) fibrosis, pulmonary fibrosis (also referred to as lung fibrosis), perivascular myocardial collagen accumulation or deposition, renal collagen accumulation or deposition, dermal (skin) collagen accumulation or deposition, pulmonary collagen accumulation or deposition, fibrotic disease or disorder associated with SLE, increased aortic media thickness, increased aortic media thickness associated with fibrosis, or any combination thereof.

Examples of cardiac diseases or disorders include, but are not limited to, cardiac failure, cardiac fibrosis, cystic fibrosis, cardiac disease or disorder associated with SLE, or any combination thereof.

Examples of renal diseases or disorders include, but are not limited to, chronic kidney disease, renal fibrosis, renal disease or disorder associated with SLE, or disorder associated to cystic fibrosis, or any combination thereof.

Examples of dermal (skin) diseases or disorders include, but are not limited to, dermatomyositis, dermal (skin) fibrosis, dermal (skin) disease or disorder associated with dermatomyositis, or any combination thereof.

Examples of pulmonary diseases or disorders include, but are not limited to, chronic obstructive pulmonary disease (COPD), pulmonary arterial hypertension (PAH), idiopathic pulmonary arterial hypertension (iPAH), IPF, pulmonary disease or disorder associated with SLE, pulmonary inflammation, or any combination thereof.

Other examples of diseases or disorders coursing with fibrosis include, but are not limited to, BOOP, ARDS, asbestosis, accidental radiation induced lung fibrosis, therapeutic radiation induced lung fibrosis, sarcoidosis, silicosis, tuberculosis, COVID-19 associated lung fibrosis, Hermansky Pudlak syndrome, bagassosis, eosinophilic granuloma, Wegener's granulomatosis, lymphangioleiomyomatosis, cystic fibrosis, fatty liver disease, cGVHD, sclerodermatous graft-versus-host disease, NFD, Dupuytren's contracture, keloids, chronic graft rejection, scarring or wound healing abnormalities, post-operative adhesions, reactive fibrosis, disease or disorder associated with nephrotoxic agent exposure, disease or disorder associated with aminoglycoside exposure, disease or disorder associated with non-steroidal anti-inflammatory drug (NSAID) exposure, disease or disorder associated with immune-suppressant exposure, disease or disorder associated with nitiofurantoin exposure, disease or disorder associated with amiodarone exposure, disease or disorder associated with bleomycin exposure, disease or disorder associated with cyclophosphamide exposure, disease or disorder associated with methotrexate exposure, myocardial infarction, injury related tissue scarring, scarring associated with surgery, therapeutic radiation induced fibrosis, dermatomyositis (DM), disease or disorder associated with endothelial cell injury, such as autoimmune-based endothelial cell injury, or any combination thereof.

While the compounds having the structure of Formula (I)-(X) are CB₂ receptor ligands, they also have neuroprotective properties. Thus, the compositions and formulations comprising one or more compounds having the structure of Formula (I)-(X) are useful in treating neurological disorders including but not limited to stroke, migraine, cluster headaches. The compositions and formulations disclosed herein are also effective in treating certain chronic degenerative diseases that are characterized by gradual selective neuronal loss. In this connection, the present compositions and formulations are effective in the treatment of Parkinson's disease, Alzheimer's disease, amyotrophic lateral sclerosis, Huntington's chorea, and prison-associated neurodegeneration. Neuroprotection conferred by CB₂ receptor agonists could also be effective in protection and/or treatment of neurotoxic agents, such as nerve gas, as well as other insults to brain or nervous tissue by way of chemical or biological agents.

By virtue of their analgesic properties it will be recognized that the compositions and formulations according to the present invention will be useful in treating pain including peripheral, visceral, neuropathic, inflammatory and referred pain. The present compositions and formulations are also effective in cardioprotection from arrhythmia, hypertension, and myocardial ischemia. The compositions and formulations disclosed herein are also effective in the treatment of muscle spasm and tremor. The compositions and formulations disclosed herein are also effective in the treatment of various cardiac diseases or disorders and/or fibrotic diseases or disorders, such as cardiac fibrosis.

The pharmaceutical compositions and formulations described herein can be administered to a subject per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, for example, include topical, oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternatively, one may administer the compound in a local rather than systemic manner, for example, via injection of the compound directly into the area of pain, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions and formulations disclosed herein may be manufactured in a manner that is itself known, e.g., by means of conventional mixing, dissolving, granulating, dragee-making, levigating, emulsifying, encapsulating, entrapping or tableting processes.

Pharmaceutical compositions and formulations for use in accordance with the present disclosure thus may be formulated in a conventional manner using one or more physiologically acceptable carriers comprising excipients and auxiliaries, which facilitate processing of the active compounds into preparations, which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents disclosed herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art.

For oral administration, either solid or fluid unit dosage forms can be prepared. For preparing solid compositions such as tablets, the compound having the structure of Formula (I)-(X) or derivatives thereof, disclosed above herein, is mixed into formulations with conventional ingredients such as talc, magnesium stearate, dicalcium phosphate, magnesium aluminum silicate, calcium sulfate, starch, lactose, acacia, methylcellulose, and functionally similar materials as pharmaceutical diluents or carriers. For oral administration, the compounds can be also formulated readily by combining the active compounds with pharmaceutically acceptable carriers well known in the art. Such carriers enable the compounds disclosed herein to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination disclosed herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Capsules are prepared by mixing the compound with an inert pharmaceutical diluent, and filling the mixture into a hard gelatin capsule of appropriate size. Soft gelatin capsules are prepared by machine encapsulation of slurry of the compound with an acceptable vegetable oil, light liquid petrolatum or other inert oil. Fluid unit dosage forms for oral administration such as syrups, elixirs and suspensions can be prepared. The water-soluble forms can be dissolved in an aqueous vehicle together with sugar, aromatic flavoring agents and preservatives to form syrup. An elixir is prepared by using a hydro alcoholic (e. g., ethanol) vehicle with suitable sweeteners such as sugar and saccharin, together with an aromatic flavoring agent. Suspensions can be prepared with an aqueous vehicle with the aid of a suspending agent such as acacia, tragacanth, methylcellulose and the like.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Starch microspheres can be prepared by adding a warm aqueous starch solution, e.g., of potato starch, to a heated solution of polyethylene glycol in water with stirring to form an emulsion. When the two-phase system has formed (with the starch solution as the inner phase) the mixture is then cooled to room temperature under continued stirring whereupon the inner phase is converted into gel particles. These particles are then filtered off at room temperature and slurred in a solvent such as ethanol, after which the particles are again filtered off and laid to dry in air. The micro spheres can be hardened by well-known cross-linking procedures such as heat treatment or by using chemical cross-linking agents. Suitable agents include dialdehydes, including glyoxal, malondialdehyde, succinic aldehyde, adipaldehyde, glutaraldehyde and phthalaldehyde, diketones such as butadione, epichlorohydrin, polyphosphate, and borate. Dialdehydes are used to crosslink proteins such as albumin by interaction with amino groups, and diketones form schiff bases with amino groups. Epichlorohydrin activates compounds with nucleophiles such as amino or hydroxyl to an epoxide derivative.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers and/or antioxidants may be added. All formulations for oral administration should be in dosages suitable for such administration.

For buccal administration, the compositions may take the form of tablets or lozenges formulated in conventional manner.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Slow or extended-release delivery systems, including any of a number biopolymers (biological-based systems), systems employing liposomes, colloids, resins, and other polymeric delivery systems or compartmentalized reservoirs, can be utilized with the compositions described herein to provide a continuous or long-term source of therapeutic compound. Such slow-release systems are applicable to formulations for delivery via topical, intraocular, oral, and parenteral routes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethyl cellulose, sorbitol, or dextran. Optionally, the suspension may also contain suitable stabilizers or agents, which increase the solubility of the compounds to allow for the preparation of highly, concentrated solutions.

Alternatively, the active ingredient may be in powder form for constitution with a suitable vehicle, e.g., sterile pyrogen-free water, before use.

In addition to the formulations described previously, the compounds may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds disclosed herein is a co-solvent system comprising benzyl alcohol, a nonpolar surfactant, a water-miscible organic polymer, and an aqueous phase. A common co-solvent system used is a co-solvent system, comprising a solution of 3% w/v benzyl alcohol, 8% w/v of the nonpolar surfactant Polysorbate 80, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of Polysorbate 80; the fraction size of polyethylene glycol may be varied; other biocompatible polymers may replace polyethylene glycol, e.g., polyvinyl pyrrolidone; and other sugars or polysaccharides may be used.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for stabilization may be employed.

Many of the compounds used in the pharmaceutical combinations disclosed herein may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acids or base forms.

Pharmaceutical compositions suitable for use in the methods disclosed herein include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions disclosed herein can be chosen by the individual physician in view of the patient's condition. (See e.g., Fingl et al. 1975, in “The Pharmacological Basis of Therapeutics”, Ch. 1 p. 1). Typically, the dose about the composition administered to the patient can be from about 0.5 to 1000 mg/kg of the patient's body weight, or 1 to 500 mg/kg, or 10 to 500 mg/kg, or 50 to 100 mg/kg of the patient's body weight. The dosage may be a single one or a series of two or more given in the course of one or more days, as is needed by the patient. Note that for almost all of the specific compounds mentioned in the present disclosure, human dosages for treatment of at least some condition have been established. Thus, in most instances, the methods disclosed herein will use those same dosages, or dosages that are between about 0.1% and 500%, or between about 25% and 250%, or between 50% and 100% of the established human dosage. Where no human dosage is established, as will be the case for newly discovered pharmaceutical compounds, a suitable human dosage can be inferred from ED50 or ID50 values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 2000 mg of each ingredient, preferably between 1 mg and 250 mg, e.g., 5 to 200 mg or an intravenous, subcutaneous, or intramuscular dose of each ingredient between 0.01 mg and 500 mg, preferably between 0.1 mg and 60 mg, e.g., 0.1 to 40 mg of each ingredient of the pharmaceutical compositions disclosed herein or a pharmaceutically acceptable salt thereof calculated as the free base, the composition being administered 1 to 4 times per day. Alternatively, the compositions disclosed herein may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 500 mg. Suitably the compounds will be administered for a period of continuous therapy, for example for a week or more, or for months or years.

Dosage amount and interval may be adjusted individually to provide plasma levels of the active moiety, which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen, which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The pharmaceutical compositions and formulations may be prepared with pharmaceutically acceptable excipients, which may be a carrier or a diluent, as a way of example. Such compositions can be in the form of a capsule, sachet, paper or other container. In making the compositions, conventional techniques for the preparation of pharmaceutical compositions may be used. For example, the compounds having the structure of Formula (I)-(X) disclosed above herein may be mixed with a carrier, or diluted by a carrier, or enclosed within a carrier that may be in the form of an ampoule, capsule, sachet, paper, or other container. When the carrier serves as a diluent, it may be solid, semi-solid, or liquid material that acts as a vehicle, excipient, or medium for the active compound. The compounds having the structure of Formula (I)-(X) and compositions comprising the same, for use as described above herein can be adsorbed on a granular solid container for example in a sachet. Some examples of suitable carriers are water, salt solutions, alcohols, polyethylene glycols, polyhydroxyethoxylated castor oil, peanut oil, olive oil, lactose, terra alba, sucrose, cyclodextrin, amylose, magnesium stearate, talc, gelatin, agar, pectin, acacia, stearic acid or lower alkyl ethers of cellulose, silicic acid, fatty acids, fatty acid amines, fatty acid mono glycerides and diglycerides, pentaerythritol fatty acid esters, polyoxyethylene, hydroxymethylcellulose, and polyvinylpyrrolidone. Similarly, the carrier or diluent may include any sustained release material known in the art, such as glyceryl monostearate or glyceryl distearate, alone or mixed with a wax. Said compositions may also include wetting agents, emulsifying and suspending agents, preserving agents, sweetening agents or flavoring agents. The compositions for use in the treatment of conditions or diseases responsive to the modulation of the CB₂ receptor activity, described in present invention may be formulated so as to provide quick, sustained, or delayed release of the compounds having the structure of Formula (I)-(X) disclosed herein after administration to the patient by employing procedures well known in the art.

The pharmaceutical compositions and formulations can be sterilized and mixed, if desired, with auxiliary agents, emulsifiers, salt for influencing osmotic pressure, buffers and/or coloring substances and the like, which do not deleteriously react with the compounds disclosed above herein.

The pharmaceutical compositions and formulations may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

The compositions of the invention may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound disclosed herein formulated in a compatible pharmaceutical carrier may also be prepared, placed in an appropriate container, and labeled for treatment of an indicated condition.

The compositions of the invention may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound disclosed herein formulated in a compatible pharmaceutical carrier may also be prepared, placed in an appropriate container, and labeled for treatment of an indicated condition.

### Method of Treatment or Prevention

The disclosure also relates, in part, to methods of treating or preventing a disease or condition associated with E2F pathway, disease or condition associated with inflammation, such as systemic inflammation or pulmonary inflammation, disease or condition associated with fibroblast, such as renal fibroblast, disease or condition associated with proliferation of fibroblast, disease or condition associated with myofibroblast, such as renal myofibroblast or dermal myofibroblast, disease or condition associated with proliferation of myofibroblast, disease or condition associated with collagen, disease or condition associated with collagen accumulation or deposition, disease or condition associated with aortic media thickness, disease or condition associated with increased aortic media thickness, disease or condition associated with increased aortic media thickness by fibrosis, disease or condition associated with macrophage infiltration, disease or condition associated with fibrotic proteins, disease or condition associated with fibrotic proteins expression, disease or condition associated with E2F-dependent genes, disease or condition associated with the expression of E2F-dependent genes, disease or condition associated with at least one inflammatory gene, disease or condition associated with the expression at least one inflammatory gene, disease or condition associated with at least one profibrotic gene, disease or condition associated with the expression of at least one profibrotic gene, fibrotic disease or disorder, autoimmune disease or disorder, such as SLE, inflammatory-related disease or disorder, cardiac disease or disorder, cardiovascular disease or disorder, renal disease or disorder, dermal (skin) disease or disorder, pulmonary disease or disorder (also referred to as lung disease or disorder), disease or disorder associated with autoimmune disease or disorder, such as a disease or disorder associated with SLE, or any combination thereof. In one embodiment, the method comprises administering to the subject in need thereof a therapeutically effective amount of at least one cannabidiol aminoquinone derivative or a composition thereof.

In one aspect, the disclosure provides a method of treating or preventing a fibrotic disease or disorder in a subject in need thereof, comprising administering a therapeutically effective amount of at least one cannabidiol aminoquinone derivative or a composition thereof to the subject. In some embodiments, the fibrotic disease or disorder is a renal fibrosis, cardiac fibrosis, cardiovascular fibrosis, aortic fibrosis, dermal (skin) fibrosis, pulmonary fibrosis, perivascular myocardial collagen accumulation or deposition, renal collagen accumulation or deposition, dermal (skin) collagen accumulation or deposition, pulmonary collagen accumulation or deposition, increased aortic media thickness, increased aortic media thickness associated with fibrosis, IPF, or any combination thereof.

It is understood that the disclosed methods of treating fibrosis can treat any fibrotic condition regardless of whether the fibrosis is the result of disease, accidental exposure to radiation, accidental tissue injury, therapeutic exposure to radiation, or surgical procedures. Thus, it is understood that the disclosed methods can be used to treat fibrosis, wherein the cause of the fibrosis includes, but is not limited to pulmonary fibrosis, IPF, BOOP, DM, ARDS, asbestosis, accidental radiation induced lung fibrosis, therapeutic radiation induced lung fibrosis, sarcoidosis, silicosis, tuberculosis, COVID-19 associated lung fibrosis, Hermansky Pudlak syndrome, bagassosis, SLE, eosinophilic granuloma, Wegener's granulomatosis, lymphangioleiomyomatosis, cystic fibrosis, fatty liver disease, cGVHD, sclerodermatous graft versus host disease, NFD, nephrotoxic agent exposure, aminoglycoside exposure, NSAID exposure, immune-suppressant exposure, nitrofurantoin exposure, amiodarone exposure, bleomycin exposure, cyclophosphamide exposure, methotrexate exposure, DM, endothelial cell injury, such as autoimmune-based endothelial cell injury, myocardial infarction, injury related tissue scarring, scarring form surgery, therapeutic radiation induced fibrosis, or any combination thereof.

In one aspect, the disclosure provides a method of treating or preventing a cardiac disease or disorder in a subject in need thereof, comprising administering a therapeutically effective amount of at least one cannabidiol aminoquinone derivative or a composition thereof to the subject. In some embodiments, the cardiac disease or disorder is a cardiac failure, cardiac fibrosis, cardiac disease or disorder associated with SLE, or any combination thereof.

In one aspect, the disclosure provides a method of treating or preventing a renal disease or disorder in a subject in need thereof, comprising administering a therapeutically effective amount of at least one cannabidiol aminoquinone derivative or a composition thereof to the subject. In some embodiments, the renal disease or disorder is a kidney disease or disorder, acute kidney disease or disorder, chronic kidney disease or disorder, end stage renal disease (ESRD), renal fibrosis, renal disease or disorder associated with SLE, or any combination thereof.

In one aspect, the disclosure provides a method of treating or preventing a dermal disease or disorder in a subject in need thereof, comprising administering a therapeutically effective amount of at least one cannabidiol aminoquinone derivative or a composition thereof to the subject. In some embodiments, the dermal disease or disorder is a dermal fibrosis, dermatomyositis, dermal (skin) disease or disorder associated with dermatomyositis, dermal inflammation, or any combination thereof.

In one aspect, the disclosure provides a method of treating or preventing a pulmonary disease or disorder in a subject in need thereof, comprising administering a therapeutically effective amount of at least one cannabidiol aminoquinone derivative or a composition thereof to the subject. In some embodiments, the pulmonary disease or disorder is a COPD, PAH, iPAH, IPF, pulmonary disease or disorder associated with SLE, pulmonary inflammation, cystic fibrosis, or any combination thereof.

In some aspects, the present disclosure further relates, in part, to a method of modulating the activity of E2F pathway, level or activity of at least one E2F-dependent gene, level of myofibroblast, level of fibroblast, level of fibrosis, level of macrophage accumulation, level of macrophage infiltration, level of collagen accumulation or deposition, aortic media thickness, aortic media thickness associated with fibrosis, level or activity of inflammatory response, level or activity of at least one fibrotic protein, level or activity of interferon response, level or activity of IFN-α response, level or activity of IFN-γ response, immune response associated with fibrosis, autoimmune response associated with fibrosis, inflammation associated with fibrosis, or any combination thereof in a subject in need thereof. In various embodiments, the method comprises administering a therapeutically effective amount of at least one cannabidiol aminoquinone derivative or a composition thereof to the subject.

For example, in some embodiments, the present disclosure relates, in part, to a method of modulating the interstitial collagen accumulation or deposition, interstitial collagen accumulation or deposition in cardiac tissue, myocardial collagen accumulation or deposition, perivascular myocardial collagen accumulation or deposition, renal collagen accumulation or deposition, dermal (skin) collagen accumulation or deposition, pulmonary collagen accumulation or deposition, or any combination thereof in a subject in need thereof by administering a therapeutically effective amount of at least one cannabidiol aminoquinone derivative or a composition thereof to the subject.

In some embodiments, the present disclosure relates, in part, to a method of modulating the level (e.g., expression, activity, level, etc.) of at least one gene in a subject in need thereof by administering a therapeutically effective amount of at least one cannabidiol aminoquinone derivative or a composition thereof to the subject. For example, in one embodiment, the disclosure comprises a method of modulating the level (e.g., expression, activity, level, etc.) of at least one E2F-dependent gene in a subject in need thereof by administering a therapeutically effective amount of at least one cannabidiol aminoquinone derivative or a composition thereof to the subject. In some embodiments, the disclosure comprises a method of modulating the level (e.g., expression, activity, level, etc.) of CDK1 gene, TOP2A gene, MKi67 gene, IL6 gene, IL1β gene, TNC gene, inflammatory gene, profibrotic gene, or any combination thereof.

In one embodiment, the method of the present disclosure comprises a cannabidiol aminoquinone derivative or composition thereof that further modulates at least one gene expression. In one embodiment, the cannabidiol aminoquinone derivative or composition thereof further prevents at least one gene expression. In one embodiment, the method of the present disclosure comprises a cannabidiol aminoquinone derivative or composition thereof that further reduces at least one gene expression. In one embodiment, the method of the present disclosure comprises a cannabidiol aminoquinone derivative or composition thereof that further inhibits at least one gene expression. For examples, in some embodiments, the method comprises a cannabidiol aminoquinone derivative or composition thereof that modulates a gene expression associated with E2F.

In other embodiments, the method comprises a cannabidiol aminoquinone derivative or composition thereof that modulates the level (e.g., expression, activity, level, etc.) of CDK1, TOP2A, MKi67, IL6, IL1β, TNC, or any combination thereof. In some embodiments, the method comprises a cannabidiol aminoquinone derivative or composition thereof that reduces the level (e.g., expression, activity, level, etc.) of CDK1, TOP2A, MKi67, IL6, IL1β, TNC, or any combination thereof. In some embodiments, the method comprises a cannabidiol aminoquinone derivative or composition thereof that inhibits the level (e.g., expression, activity, level, etc.) of CDK1, TOP2A, MKi67, IL6, IL1β, TNC, or any combination thereof.

In some aspects, the present disclosure relates, in part, to a method of reducing the activity of E2F pathway, level or activity of at least one E2F-dependent gene, level of myofibroblast, level of fibroblast, level of fibrosis, level of macrophage accumulation, level of macrophage infiltration, level of collagen accumulation or deposition, aortic media thickness, aortic media thickness associated with fibrosis, level or activity of inflammatory response, level or activity of at least one fibrotic protein, level or activity of interferon response, level or activity of IFN-α response, level or activity of IFN-γ response, immune response associated with fibrosis, autoimmune response associated with fibrosis, inflammation associated with fibrosis, or any combination thereof.

In other aspects, the present disclosure relates, in part, to a method of inhibiting the activity of E2F pathway, activity of at least one E2F-dependent gene, proliferation of myofibroblast, proliferation of fibroblast, proliferation of fibrosis, macrophage accumulation, macrophage infiltration, collagen accumulation or deposition, development of aortic media thickness, development of aortic media thickness associated with fibrosis, activity of inflammatory response, activity of at least one fibrotic protein, activity of interferon response, activity of IFN-α response, activity of IFN-γ response, immune response associated with fibrosis, autoimmune response associated with fibrosis, inflammation associated with fibrosis, and any combination thereof.

In one aspect, the present disclosure further relates to a method of treating a disease or condition responsive to a modulation of E2F activity (e.g., level, activity, expression, etc.) in a subject in need thereof by administering a therapeutically effective amount of a cannabidiol aminoquinone derivative or composition thereof to the subject.

In one embodiment, the cannabidiol aminoquinone derivative or composition thereof is a modulator of E2F activity (e.g., level, activity, expression, etc.). Thus, for example, in one embodiment, the cannabidiol aminoquinone derivative or composition thereof reduces fibrosis by acting on E2F activity (e.g., level, activity, expression, etc.).

In some embodiments, the cannabidiol aminoquinone derivative or composition thereof decreased α-SMA expression in cultured human immortalized cardiac fibroblasts (hICF) treated with either TGF-β or Ang II, and inhibited both Ang II-induced ERK1+2 phosphorylation and NF-AT activation in hICF. Also, the cannabidiol aminoquinone derivative or composition thereof inhibited the mRNA expression of IL1b, IL6, Col1A2 and CCL2, induced by Ang II or TGF-β in hICF cells.

In another embodiment, the cannabidiol aminoquinone derivative or composition thereof is a modulator of PPARγ and E2F activity (e.g., level, activity, expression, etc.). In one embodiment, the cannabidiol aminoquinone derivative or composition thereof is a dual modulator of PPARγ and E2F activity (e.g., level, activity, expression, etc.), and it activates the HIF pathway. In one embodiment, the cannabidiol aminoquinone derivative or composition thereof is a triple modulator of PPARγ, CB₂, and E2F activity (e.g., level, activity, expression, etc.), and it activates the HIF pathway. In one embodiment, the cannabidiol aminoquinone derivative or composition thereof is a triple modulator of PPARγ, CB₂, and E2F activity (e.g., level, activity, expression, etc.), and it activates the HIF pathway by stabilizing HIF-1α and upregulates the expression of several associated factors that include Erythropoietin (EPO) and Vascular Endothelial Growth Factor A (VEGFA). Thus, in one embodiment, the cannabidiol aminoquinone derivative or composition thereof reduces fibrosis by acting on PPARγ, CB₂, E2F activity (e.g., level, activity, expression, etc.), HIF pathway, or any combination thereof.

In some embodiments, the disease or condition responsive to a modulation of E2F activity (e.g., level, activity, expression, etc.) is a fibrotic disease or disorder, including, but not limited to cardiac fibrosis, aortic fibrosis, dermal (skin) fibrosis, pulmonary fibrosis, renal fibrosis, and fibrosis associated with SLE, cardiac disease or disorder, cardiovascular disease or disorder, autoimmune disease or disorder, including, but not limited to SLE, dermal (skin) disease or disorder, including, but not limited to dermatomyositis, dermal (skin) disease or disorder associated with dermatomyositis, and dermal inflammation, renal disease or disorder, including, but not limited to chronic kidney disease and renal disease or disorder associated with SLE, pulmonary disease or disorder, including, but not limited to IPF, COPD, PAH, iPAH, cystic fibrosis, and pulmonary inflammation, or any combination thereof.

In another embodiment, the subject has a condition or disease responsive to the modulation of the CB₂ receptor activity. In one embodiment, the subject has a condition or disease responsive to the modulation of the E2F transcriptional signature and a condition or disease responsive to the modulation of the CB₂ receptor activity.

In some embodiments, the condition or disease responsive to the modulation of the E2F transcriptional signature is autoimmune disease, demyelinating disease, inflammatory-related disorder, SSc, myelinoclastic disorder, analgesia, acute and chronic pain, inflammatory pain, post-operative pain, neuropathic pain, muscle relaxation, immunosuppression, as anti-inflammatory agents, for allergies, glaucoma, bronchodilation, neuroprotection, osteoporosis and disorders of the skeletal system, cancer, neurodegenerative disorders including but not limited to Alzheimer's disease, Parkinson's disease (PD), and Huntington's disease, MS, muscle spasticity, tremor, fibromyalgia, lupus, rheumatoid arthritis, myasthenia gravis, other autoimmune disorders, irritable bowel syndrome, interstitial cystitis, migraine, pruritis, eczema, seborrhea, psoriasis, shingles, cerebral ischemia, cerebral apoplexy, craniocerebral trauma, stroke, spinal cord injury, liver cirrhosis, liver fibrosis, atherosclerosis, as an anti-tussive, asthma, nausea, emesis, gastric ulcers, neuromyelitis optica, central nervous system neuropathy, central pontine myelinolysis, myelopathy, leukoencephalopathy, leukodystrophy, peripheral neuropathy, Guillain-Barre syndrome, anti-MAG peripheral neuropathy, Charcot-Marie-Tooth disease, progressive inflammatory neuropathy, amyotrophic lateral sclerosis (ALS), or any combination thereof.

In one aspect, the present invention relates to new drug candidates comprising chemically stable, nonpsychotropic aminoquinoid chemically derived from synthetic or natural cannabidiol (CBD) through oxidation and amination. In some embodiments, the synthetic cannabidiol derivative of the present disclosure has a novel mechanism of action by targeting complementary signaling pathways that alleviate neuroinflammation and favor neuroprotection, prevent axonal damage, preserve myelin structure, modulates the CB₂ activity, modulates the E2F transcriptional signature, and potentially promotes remyelination. In one embodiment, the synthetic cannabidiol derivative of the present invention is a modulator of CB₂ receptor signaling. In one embodiment, the synthetic cannabidiol derivative of the present invention is a modulator of the E2F transcriptional signature. In one embodiment, the synthetic cannabidiol derivative of the present invention is a modulator of PPARγ and CB₂ receptor signaling. In one embodiment, the synthetic cannabidiol derivative of the present invention is a modulator of PPARγ, CB₂ receptor signaling, and E2F transcriptional signature. In one embodiment, the synthetic cannabidiol derivative of the present invention is a dual modulator of PPARγ and CB₂ receptor signaling, and it activates the HIF pathway by stabilizing HIF-1α and upregulates the expression of several associated factors that include Erythropoietin (EPO) and Vascular Endothelial Growth Factor A (VEGFA). In one embodiment, the synthetic cannabidiol derivative of the present invention is a modulator of PPARγ, CB₂ receptor signaling, and E2F transcriptional signature and it activates the HIF pathway by stabilizing HIF-1α and upregulates the expression of several associated factors that include Erythropoietin (EPO) and Vascular Endothelial Growth Factor A (VEGFA). In one embodiment, the synthetic cannabidiol derivative of the present invention reduces neuroinflammation presumably by acting on PPARγ/CB₂ receptors, in conjunction with enhanced neuroprotection and potential remyelination through the HIF pathway.

CB₂ modulators (i.e., agonists, partial agonists, antagonists, or inverse agonists) have therapeutic utility for analgesia, acute and chronic pain, inflammatory pain, post-operative pain, neuropathic pain, muscle relaxation, immunosuppression, as anti-inflammatory agents, for allergies, glaucoma, bronchodilation, neuroprotection, osteoporosis and disorders of the skeletal system, cancer, neurodegenerative disorders including but not limited to Alzheimer's disease, Parkinson's disease (PD), and Huntington's disease, multiple sclerosis (MS), muscle spasticity, tremor, fibromyalgia, lupus, rheumatoid arthritis, myasthenia gravis, other autoimmune disorders, irritable bowel syndrome, interstitial cystitis, migraine, pruritis, eczema, sebhorea, psoriasis, shingles, cerebral ischemia, cerebral apoplexy, craniocerebral trauma, stroke, spinal cord injury, liver cirrhosis, liver fibrosis, atherosclerosis, as an anti-tussive, asthma, nausea, emesis, gastric ulcers, systemic sclerosis, myelinoclastic disorder, neuromyelitis optica, central nervous system neuropathy, central pontine myelinolysis, myelopathy, leukoencephalopathy, leukodystrophy, peripheral neuropathy, Guillain-Barre syndrome, anti-MAG peripheral neuropathy, Charcot-Marie-Tooth disease, progressive inflammatory neuropathy, fibrotic disease, cardiac fibrosis, cardiovascular fibrosis, perivascular myocardial collagen deposition, increased aortic media thickness, amyotrophic lateral sclerosis (ALS), and diarrhea.

Thus, in one aspect, the present invention further relates to a method of treating a disease or condition responsive to the modulation of CB₂ receptor activity in a subject. In one embodiment, the method comprises identifying a subject in need thereof and administering to the subject a therapeutically effective amount of the synthetic cannabidiol derivative of the present invention or the composition thereof.

In one embodiment, the synthetic cannabidiol derivative of the present invention modulates the activity of a CB₂. In one embodiment, the synthetic cannabidiol derivative of the present invention preferentially binds to CB₂ receptor as compared to CB₁. Therefore, in these embodiments, the synthetic cannabidiol derivative of the present invention is selective for CB₂. In one embodiment, the amine group of the synthetic cannabidiol derivative of the present invention enhances its binding to the CB₂. In one embodiment, the amine group of the synthetic cannabidiol derivative of the present invention selectively binds the CB2 receptor over the CB₁ receptor. In one embodiment, the CB₂ receptor activity is modulated in vitro, whereas in other embodiments, the CB₂ receptor activity is modulated in vivo.

In some embodiments, the condition or disease responsive to the modulation of the CB₂ receptor activity is autoimmune disease, demyelinating disease, inflammatory-related disorder, cardiac disease or disorder, cardiovascular disease or disorder, or any combination thereof. In some embodiments, the condition or disease responsive to the modulation of the CB₂ receptor activity is fibrotic disease, cardiac fibrosis, cardiovascular fibrosis, perivascular myocardial collagen deposition, increased aortic media thickness, SSc, myelinoclastic disorder, analgesia, acute and chronic pain, inflammatory pain, post-operative pain, neuropathic pain, muscle relaxation, immunosuppression, as anti-inflammatory agents, for allergies, glaucoma, bronchodilation, neuroprotection, osteoporosis and disorders of the skeletal system, cancer, neurodegenerative disorders including but not limited to Alzheimer's disease, Parkinson's disease (PD), and Huntington's disease, MS, muscle spasticity, tremor, fibromyalgia, lupus, rheumatoid arthritis, myasthenia gravis, other autoimmune disorders, irritable bowel syndrome, interstitial cystitis, migraine, pruritis, eczema, sebhorea, psoriasis, shingles, cerebral ischemia, cerebral apoplexy, craniocerebral trauma, stroke, spinal cord injury, liver cirrhosis, liver fibrosis, atherosclerosis, as an anti-tussive, asthma, nausea, emesis, gastric ulcers, neuromyelitis optica, central nervous system neuropathy, central pontine myelinolysis, myelopathy, leukoencephalopathy, leukodystrophy, peripheral neuropathy, Guillain-Barre syndrome, anti-MAG peripheral neuropathy, Charcot-Marie-Tooth disease, progressive inflammatory neuropathy, amyotrophic lateral sclerosis (ALS), or any combination thereof.

In one embodiment, the cannabidiol aminoquinone derivative or composition thereof is administered in combination with another therapeutic agent. In one embodiment, the cannabidiol aminoquinone derivative or composition thereof is administered orally. In one embodiment, the cannabidiol aminoquinone derivative or composition thereof is administered topically. In one embodiment, the cannabidiol aminoquinone derivative or composition thereof is administered using rectal administration. In one embodiment, the cannabidiol aminoquinone derivative or composition thereof is administered using transmucosal administration. In one embodiment, the cannabidiol aminoquinone derivative or composition thereof is administered using intestinal administration. In one embodiment, the cannabidiol aminoquinone derivative or composition thereof is administered using parenteral delivery. In one embodiment, the cannabidiol aminoquinone derivative or composition thereof is administered using intramuscular injection. In one embodiment, the cannabidiol aminoquinone derivative or composition thereof is administered using subcutaneous injection. In one embodiment, the cannabidiol aminoquinone derivative or composition thereof is administered using intravenous injection. In one embodiment, the cannabidiol aminoquinone derivative or composition thereof is administered using intramedullary injection. In one embodiment, the cannabidiol aminoquinone derivative or composition thereof is administered using intrathecal injection. In one embodiment, the cannabidiol aminoquinone derivative or composition thereof is administered using direct intraventricular injection. In one embodiment, the cannabidiol aminoquinone derivative or composition thereof is administered using intraperitoneal injection. In one embodiment, the cannabidiol aminoquinone derivative or composition thereof is administered using intranasal injection. In one embodiment, the cannabidiol aminoquinone derivative or composition thereof is administered using intraocular injection.

In one aspect of the invention, the method of treating a condition or disease responsive to the modulation of the E2F transcriptional signature further comprises modulating remyelination.

In another aspect of the invention, the method of treating a condition or disease responsive to the modulation of the CB₂ receptor activation further comprises modulating remyelination.

Thus, in one aspect, the present invention also relates, in part, to a method of treating demyelination diseases. In one embodiment, the synthetic cannabidiol derivative of the present invention is effective for the attenuation of demyelination in a subject.

By “attenuation of demyelination” it is meant that the amount of demyelination in the subject as a result of the disease or as a symptom of the disease is reduced when compared to otherwise same conditions and/or the amount of remyelination in the subject is increased when compared to otherwise same conditions.

By “reduced” it is meant any measurable or detectable reduction in the amount of demyelination or in any symptom of the demyelination disease that is attributable to demyelination.

Likewise, the term “increased” means any measurable or detectable increase in the amount of remyelination which will also manifest as a reduction in any symptom of the demyelination disease that is attributable to demyelination. In an embodiment of the invention, attenuation of demyelination in a subject is as compared to a control. Symptoms attributable to demyelination will vary depending on the disease but may include, for example but not limited to, neurological deficits, such as cognitive impairment (including memory, attention, conceptualization and problem-solving skills) and information processing; paresthesias in one or more extremities, in the trunk, or on one side of the face; weakness or clumsiness of a leg or hand; or visual disturbances, e.g., partial blindness and pain in one eye (retrobulbar optic neuritis), dimness of vision, or scotomas. The ability of a compound to attenuate demyelination may be detected or measured using assays known in the art, for example, the cuprizone-induced demyelination models described herein.

In one embodiment, the synthetic cannabidiol derivative of the present invention modulates remyelination. In one embodiment, the synthetic cannabidiol derivative of the present invention induces remyelination. In one embodiment, the synthetic cannabidiol derivative of the present invention enhances remyelination. In one embodiment, the synthetic cannabidiol derivative of the present invention modulates demyelination. In one embodiment, the synthetic cannabidiol derivative of the present invention prevents demyelination. In one embodiment, the synthetic cannabidiol derivative of the present invention reduces demyelination. In one embodiment, the synthetic cannabidiol derivative of the present invention accelerates demyelination. In one embodiment, the synthetic cannabidiol derivative of the present invention terminates demyelination. In one embodiment, the synthetic cannabidiol derivative of the present invention modulates neuroinflammation. In one embodiment, the synthetic cannabidiol derivative of the present invention alleviates neuroinflammation. In one embodiment, the synthetic cannabidiol derivative of the present invention modulates microgliosis. In one embodiment, the synthetic cannabidiol derivative of the present invention prevents microgliosis. In one embodiment, the synthetic cannabidiol derivative of the present invention alleviates microgliosis. In one embodiment, the synthetic cannabidiol derivative of the present invention modulates astrogliosis. In one embodiment, the synthetic cannabidiol derivative of the present invention prevents astrogliosis. In one embodiment, the synthetic cannabidiol derivative of the present invention alleviates astrogliosis.

In one embodiment, the demyelination disease is any disease or condition that results in damage to the protective covering (myelin sheath) that surrounds nerves in the brain and spinal cord. In a further embodiment of the invention, the demyelination disease is selected from multiple sclerosis, transverse myelitis, Guillain Barre syndrome, progressive multifocal leukoencephalopathy, transverse myelitis, phenylketonuria and other aminoacidurias, Tay-Sachs disease, Niemann-Pick disease, Gaucher's diseases, Hurler's syndrome, Krabbe's disease and other leukodystrophies, acute disseminated encephalomyelitis (postinfectious encephalomyelitis, adrenoleukodystrophy, adrenomyeloneuropathy, optic neuritis. Devic disease (neuromyelitis optica), Leber's hereditary optic atrophy and related mitochondrial disorders and HTLV-associated myelopathy or the demyelination disease is a result of local injury, ischemia, toxic agents, or metabolic disorders. In one embodiment, the demyelination disease is multiple sclerosis.

In one embodiment, the synthetic cannabidiol derivative of the present invention modulates a gene expression. In one embodiment, the synthetic cannabidiol derivative of the present invention prevents a gene expression. In one embodiment, the synthetic cannabidiol derivative of the present invention reduces a gene expression. In one embodiment, the synthetic cannabidiol derivative of the present invention enhances a gene expression.

In some embodiments, the synthetic cannabidiol derivative of the present invention modulates a gene expression selected from the group consisting of a gene associated with MS pathophysiology, a gene associated with oligodendrocyte function, a gene associated with downregulation in EAE, a gene associated with expression of Olig2, and any combination thereof. In one embodiment, the synthetic cannabidiol derivative of the present invention modulates an expression of Teneurin. In one embodiment, the synthetic cannabidiol derivative of the present invention modulates an expression of Teneurin 4 (Tenm 4). In one embodiment, the synthetic cannabidiol derivative of the present invention enhances an expression of Tenm 4. In one embodiment, the synthetic cannabidiol derivative of the present invention normalizes an expression of Tenm 4. In one embodiment, the synthetic cannabidiol derivative of the present invention modulates an expression of Olig2. In one embodiment, the synthetic cannabidiol derivative of the present invention restores an expression of Olig2. In one embodiment, the synthetic cannabidiol derivative of the present invention enhances an expression of Olig2. In one embodiment, the synthetic cannabidiol derivative of the present invention modulates an expression of glutathione S-transferase pi (GSTpi). In one embodiment, the synthetic cannabidiol derivative of the present invention enhances an expression of GSTpi. In one embodiment, the synthetic cannabidiol derivative of the present invention restores an expression of GSTpi.

In one embodiment, the synthetic cannabidiol derivative of the present invention or the composition thereof is administered in combination with another therapeutic agent. In one embodiment, the synthetic cannabidiol derivative of the present invention or the composition thereof is administered orally. In one embodiment, the synthetic cannabidiol derivative of the present invention or the composition thereof is administered topically. In one embodiment, the synthetic cannabidiol derivative of the present invention or the composition thereof is administered using rectal administration. In one embodiment, the synthetic cannabidiol derivative of the present invention or the composition thereof is administered using transmucosal administration. In one embodiment, the synthetic cannabidiol derivative of the present invention or the composition thereof is administered using intestinal administration. In one embodiment, the synthetic cannabidiol derivative of the present invention or the composition thereof is administered using parenteral delivery. In one embodiment, the synthetic cannabidiol derivative of the present invention or the composition thereof is administered using intramuscular injection. In one embodiment, the synthetic cannabidiol derivative of the present invention or the composition thereof is administered using subcutaneous injection. In one embodiment, the synthetic cannabidiol derivative of the present invention or the composition thereof is administered using intravenous injection. In one embodiment, the synthetic cannabidiol derivative of the present invention or the composition thereof is administered using intramedullary injection. In one embodiment, the synthetic cannabidiol derivative of the present invention or the composition thereof is administered using intrathecal injection. In one embodiment, the synthetic cannabidiol derivative of the present invention or the composition thereof is administered using direct intraventricular injection. In one embodiment, the synthetic cannabidiol derivative of the present invention or the composition thereof is administered using intraperitoneal injection. In one embodiment, the synthetic cannabidiol derivative of the present invention or the composition thereof is administered using intranasal injection. In one embodiment, the synthetic cannabidiol derivative of the present invention or the composition thereof is administered using intraocular injection.

In one embodiment, the cannabidiol aminoquinone derivative or composition thereof is administered with food or drink.

It will be understood by those of skill in the art that numerous and various modifications can be made without departing from the spirit of the present disclosure. Therefore, it should be clearly understood that the forms disclosed herein are illustrative only and are not intended to limit the scope of the present disclosure.

### EXPERIMENTAL EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods. The following working examples therefore, specifically point out the preferred embodiments of the present disclosure, and are not to be construed as limiting in any way the remainder of the disclosure.

### Example 1: Compounds and Compositions for the Treatment and Prevention of Cardiac, Lung (Pulmonary), Skin (Dermal), and Kidney Fibrosis

The present disclosure provides a novel application for a compound that is a cannabidiol aminoquinone derivative that exhibits activity in modulating PPARγ (e.g., see PCT Application Publication No.: WO 2015/158381 A1, disclosure of which is incorporated herein by reference in its entirety) and exhibit activity inhibiting the HIF prolyl hydroxylases (PHDs) as a consequence results in the stabilization of the HIF-1α and HIF-2α levels (e.g., see PCT Application Publication No.: WO 2018/177516, disclosure of which is incorporated herein by reference in its entirety). More specifically, the present disclosure relates to compound having the structure of Formula (VIII):

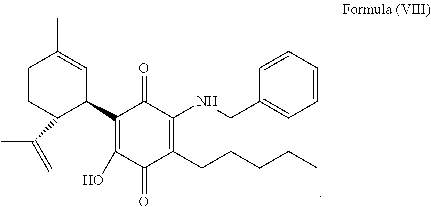

The present disclosure also provides compounds with exhibit activity regulating the E2F hallmark and as a consequence result in the regulation some markers related to this pathway, and inhibiting the CDK1, TOP2A and MKi67. The inhibition of the mentioned genes reduced the proliferation of myofibroblast. The inhibition of E2F activity in certain cardiac diseases provides a novel therapeutic approach for inhibiting the development of myocyte hypertrophy that likely delays, and possibly prevents, the onset of heart failure. In conclusion, E2F plays an important role in cardiac myocyte growth and in proliferation and differentiation on cardiac fibroblast and targeting these transcription factors provides an effective therapy for treating or preventing cardiac fibrosis. Thus, one embodiment disclosed herein refers to the use of the compound for prevent or treat cardiac fibrosis.

The term cardiac fibrosis is an abnormal deposition of ECM in the myocardium due to inappropriate proliferation of cardiac fibroblasts but more commonly refers to the proliferation of fibroblasts in the cardiac muscle and its differentiation to myofibroblast. The fibroblast normally secrete collagen, and its function is to provide structural support for the heart. But when the fibroblasts over proliferate and differentiate to myofibroblast it causes excess of collagen accumulation and subsequent fibrosis in left ventricle of the heart.

Furthermore, kidney fibrosis is characterized by accumulation of fibroblasts, which increased the production of collagen matrix deposition and attenuated EPO production. According to a preferred embodiment, the disclosure also provides a method for antagonizing collagen secretion or collagen deposition in heart and kidney using a therapeutically effective amount of a compound or composition thereof.

Dermal fibrosis is driven by immune, autoimmune, and inflammatory mechanisms. The balance of collagen production and degradation by myofibroblasts plays a critical role in the pathophysiology of fibrotic processes in the skin. Certain cytokines promote fibrosis, such as IL1β and IL6, IFN-γ, and transforming growth factor-α (TNF-α).

Besides, pulmonary fibrosis exhibits the evidence of increased numbers of activated fibroblasts, many of which have the phenotypic characteristics of myofibroblasts. (Phan S H, 2002, Chest, 122:286S-289S). Indeed, pulmonary fibrosis is a devastating lung problem manifested by excessive deposition of ECM in the lung. Fibrotic lesions distort lung architecture and alveolar structure, thicken alveolar walls, reduce lung compliance, decrease oxygen diffusion capacity and ultimately impair lung function (Gross T J et al., 2001, N. Engl. J. Med., 345:517-525).

Moreover, the compound of the present disclosure showed capacity to prevent interstitial collagen accumulation in cardiac tissue (e.g., Example 2); prevent perivascular myocardial collagen deposition perivascular (e.g., Example 2); prevent increase aortic media thickness induced by collagen accumulation (e.g., Example 2); ameliorate inflammation and macrophage infiltration in cardiac tissue (e.g., Example 3, Example 4, and Example 5); prevent of fibrotic proteins expression in left ventricle (e.g., Example 3, Example 4, and Example 5); down regulate the expression of E2F related genes pathway (e.g., Example 4 and Example 5); reduce proliferation of cardiac fibroblast by regulation E2F pathway (e.g., Example 6); prevent renal fibrosis (e.g., Example 7); prevent dermal fibrosis (e.g., Example 8); reduce perivascular myocardial collagen deposition perivascular (e.g., Example 9); reduce aortic media thickness induced by collagen accumulation (e.g., Example 9); reduce renal fibrosis (e.g., Example 9); and reduce pulmonary fibrosis (e.g., Example 10).

### Example 2: Preventive Effect of the Compound of Formula (VIII) on Cardiac Fibrosis Mouse Induced by Angiotensin II (Ang II)

Ang II causes myocardial fibrosis in a short period of time (Sopel M et al., 2011, Lab. Invest., 91:565-578) characterized by collagen accumulation and after 14 days of Ang II infusion (Sigusch H H et al., 1996, Cardiovascular Research, 31:546-554). In the preventive model, infusion of Ang II for two weeks resulted in fibrosis and increased collagen accumulation. Subcutaneous infusion of Ang II using ALZET® osmotic pumps was used to study compound preventive effects. ALZET® osmotic minipumps (Model #2004, Charles Rives, Barcelona, Spain) delivering of Ang II (1.0 mg kg⁻ min⁻) (Sigma-Aldrich, Madrid, Spain) for two weeks. Compound treatment was daily administered in parallel, one day before of the implantation of the subcutaneous pump, beneath the mid scapular loose skin and consisted of daily oral gavage of compound (20 mg/kg) or vehicle (Corn Oil/Maisine CC, 1/1). Mice were acclimated to manipulators for a week previously to the experiments in order to reduce stress and allow the detection of subtle changes in behavior by researchers. Mice weight was evaluated weakly and no significant changes were observed between experimental groups. No behavioral changes were observed during the protocols. Neither piloerection nor hunched appearances were appreciated during the experimental procedures. Mice were sacrificed by cervical dislocation and the heart, aorta, skin, and kidney were removed and processed for histological examination or were frozen for further analysis.

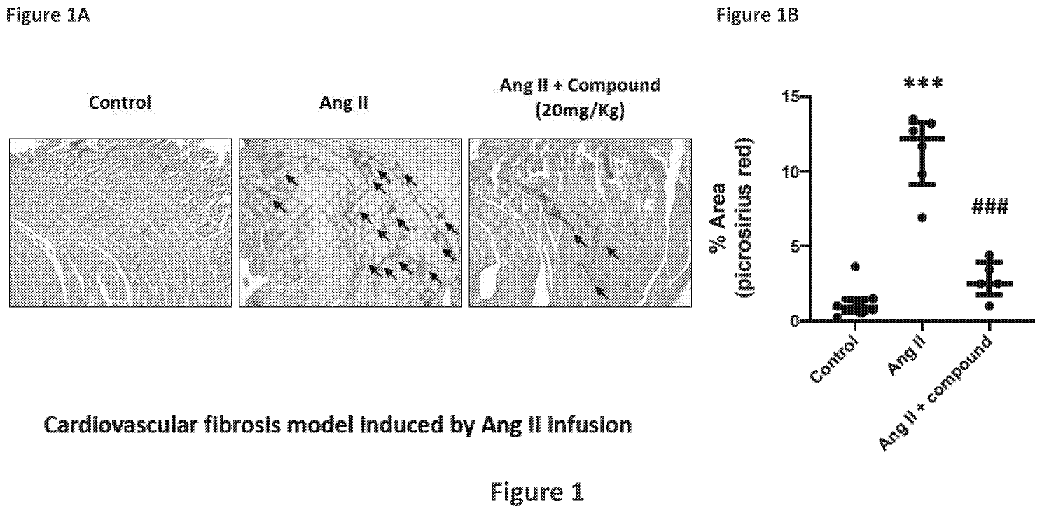

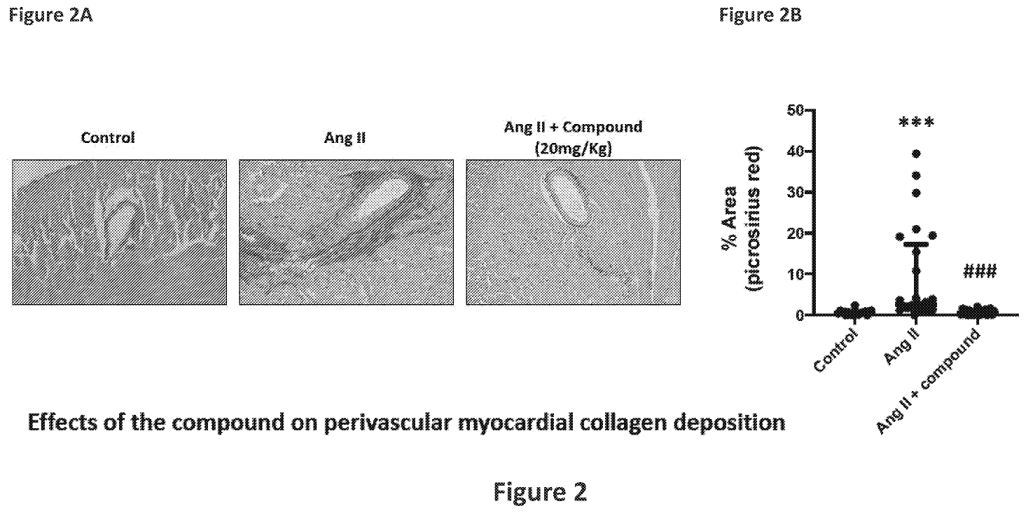

Ang II infusion for two weeks resulted in augmented on collagen accumulation versus control, which was prevented by compound treatment. Interstitial heart fibrosis was determined by the measurement of Sirius red/fast green staining (collagen content) (FIG. 1A). FIG. 1B shows that oral treatment of the compound of Formula (VIII) significantly (p<0.001) reduced the collagen content accumulation on interstitial area of left ventricle. Further analysis was undertaken to determine the collagen deposition on perivascular myocardial area by Sirius red/fast green staining. Perivascular collagen deposition content was significantly increased in heart of Ang II infused mice, as reported in FIG. 2A. Oral treatment with the compound of Formula (VIII) induced a significant reduction of perivascular collagen content (FIG. 2B).

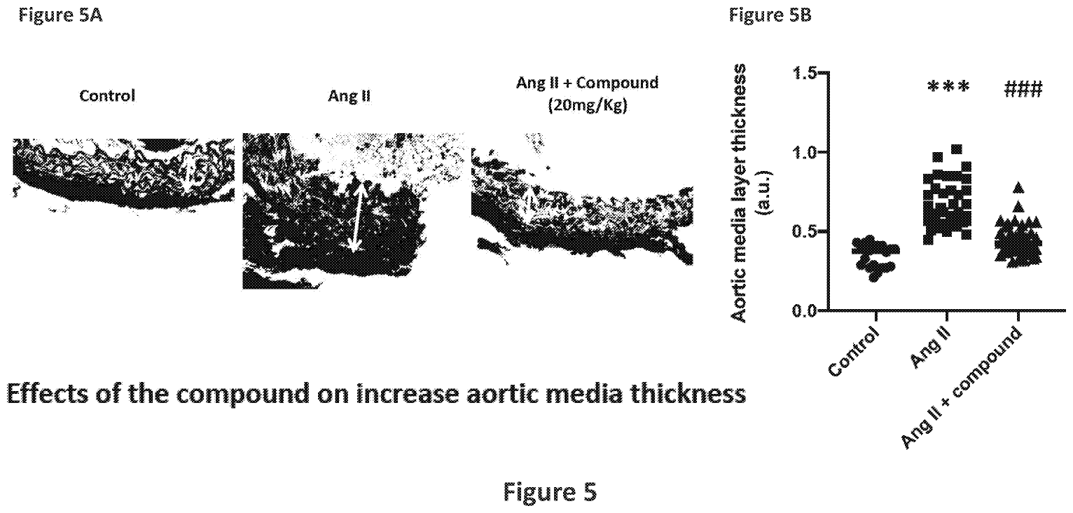

Mice treated with Ang II presented an increase in the thickness of aortic media layer by collagen accumulation. The compound of Formula (VIII) significantly alleviated the thickness of the media layer (FIG. 5). Five μm-thick tissue sections of left ventricle and aorta sections were embedded in paraffin and then stained with Sirius red/fast green staining or picrosirius red in case of the aorta. For the left ventricle, first, the sections were incubated in 0.04% Fast Green (Sigma-Aldrich, Madrid, Spain) for 15 minutes, washed with distilled water and then incubated in 0.1% Fast Green and 0.04% Sirius Red (Sigma-Aldrich, Madrid, Spain) in saturated picric acid for 30 minutes. For aorta, the sections were incubated in 0.1% Sirius Red in saturated picric acid for 60 minutes. Then, they were dehydrated and mounted with Eukitt® Mounting Medium (Sigma-Aldrich, Madrid, Spain). Collagen fibers are identified. Three-four random fields of each heart were photographed by standard brightfield microscopy and digitalized using a Leica DFC420c camera and analyzed using Fiji software (rsb.info.nih.gov/ij/).

### Example 3: Immunohistochemistry of Inflammatory and Fibrotic Markers

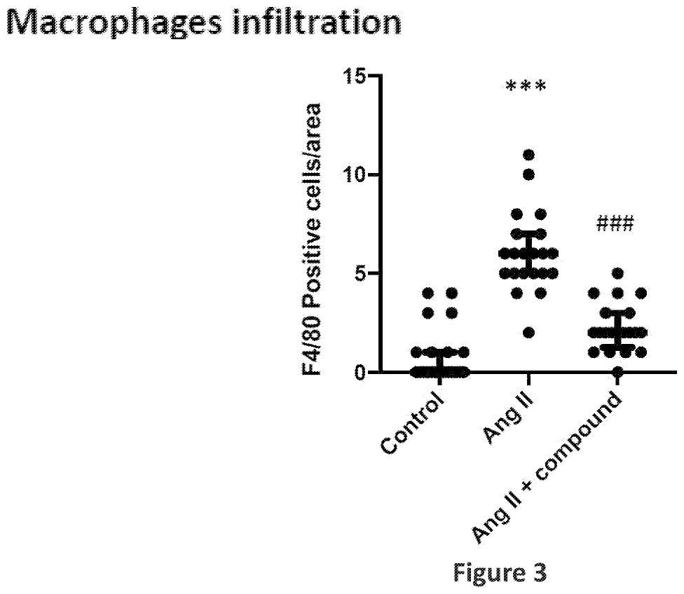

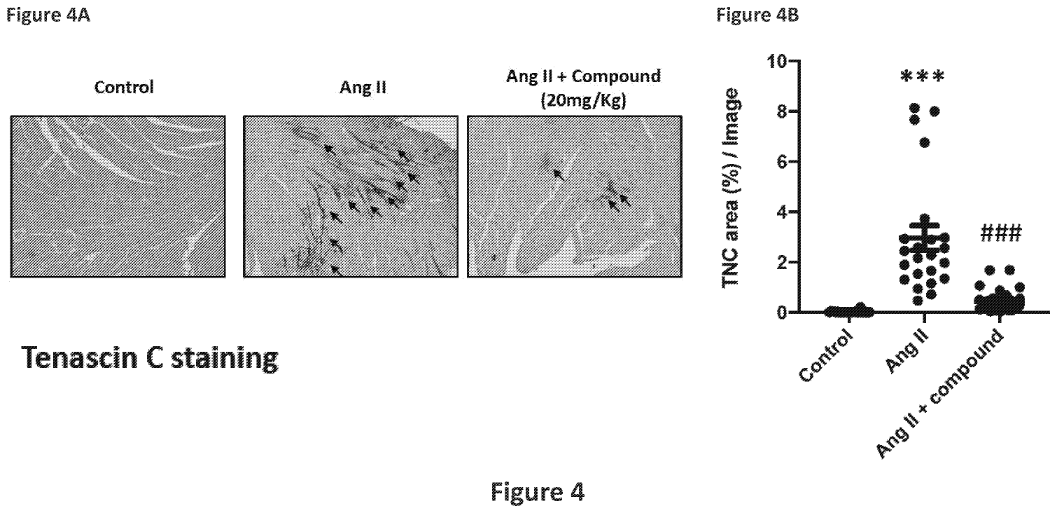

Macrophages accumulation are the hallmark of Ang II-induced fibrosis and play important roles during the inflammatory phase. Macrophage accumulation was identified by staining with a mAb that identifies F4/80⁺ cells (FIG. 3). TNC is an ECM molecule that is expressed in response to injury in various tissues. Although not detectable in the normal adult heart, it was expressed under pathological conditions (FIG. 4).

Heart sections (Left ventricle) (5 μM-thick) were deparaffinized and boiled for 10 minutes in sodium citrate buffer (10 mM, pH 6.0) (Sigma-Aldrich, Madrid, Spain) for antigen retrieval. The sections were then incubated in a PBS washing buffer containing 0.1% Tween (Sigma-Aldrich, Madrid, Spain) three times for 10 minutes. The sections were incubated overnight at 4° C. with the following antibodies; for immunohistochemical detection of macrophages F4/80 antibody (1:50; MCA497, Bio-Rad) or with monoclonal anti-TNC (1:100 dilution, #MAB2138, RD system, Mineapolis, Minn., USA) were used for detection of this ECM protein. Slides were developed with diaminobenzidine chromogen (Agilent, Santa Clara, Calif., USA) and counterstained in Harris hematoxylin (Sigma-Aldrich, Madrid, Spain). Slides were photographed, digitalized using a Leica DFC420c camera and analyzed using Image J software. Immunohistochemical examination demonstrated that the compound of Formula (VIII) significantly reduced the infiltration of F4/80+ macrophages and TNC marker compared with Ang II untreated mice (FIG. 3 and FIG. 4).

### Example 4: Compound of Formula (VIII) Modulates the Expression of E2F-Dependent Genes, Inflammation, and Fibrotic Pathway on Cardiac Tissue

RNA seq analysis from RNA extracted of myocardial tissue was performed. Transcriptome libraries were constructed with TruSeq Illumina TruSeq mRNA Sample Prep v2 kit (#RS-122-2001). In brief, 300 ng of RNA from each sample was used to construct a cDNA library, followed by sequencing on the Illumina Hiseq 2500 with single end 50 bp reads and ˜30 million reads per sample.

RNA-Seq reads were pre-processed with fastp (v0.20.0) and aligned to mouse genome assembly grcm38 using HISAT2 (v2.1.0). Then, counts per gene were obtained with featureCounts (v1.6.1) and normalization and differential expression analyses were carried out using edgeR (v3.26.8), excluding those genes with less than 15 counts across all samples. Finally, the functional analysis was performed using the functional categories defined by MSigDb hallmarks for Mus musculus (V7.0) and the clusterProfiler package (v3.12.0).

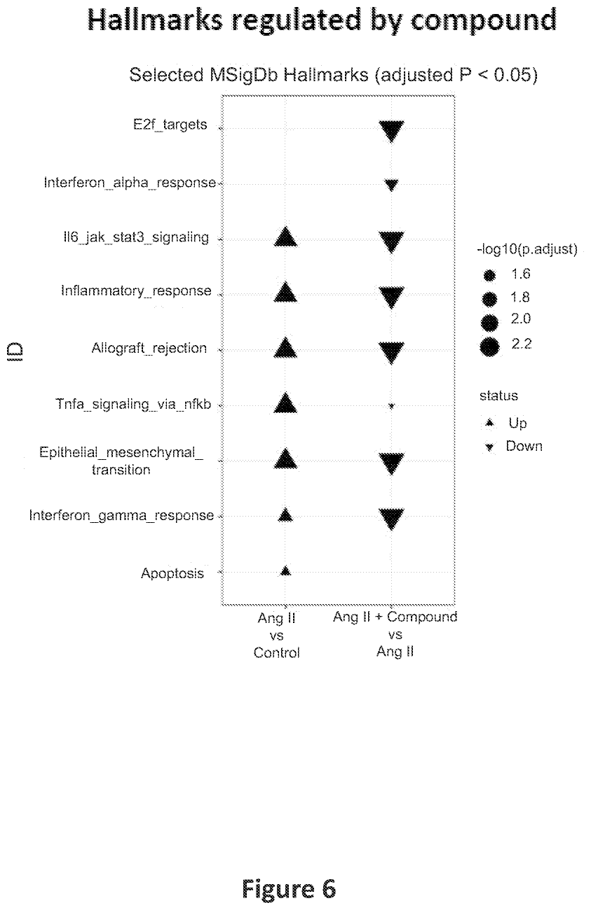

The results confirmed the hallmarks that were enriched in the Ang II group versus control group were pathways related to fibrotic response; this was the case of IFN-α and IFN-γ response, TNF-α signaling via NF-KB, Epithelial Mesenchymal Transition and inflammatory responses, including IL6 JAK STAT Signaling (FIG. 6). The results were obtained after carrying out the functional analysis of the transcriptomic signatures in heart tissue. This functional analysis consisted on a Gene Set Enrichment Analysis (GSEA, method paper: ncbi.nlm.nih.gov/pubmed/16199517) carried out using the MSigDb hallmarks as functional categories (MSigDbpaper; ncbi.nlm.nih.gov/pubmed/26771021). All the hallmarks used to generate the statement surpassed a statistical cutoff of a FDR adjusted p value <0.05 on the GSEA analysis. The analysis revealed that the hallmarks that were enriched in one direction in Ang-challenged mice followed the opposite trend after treatment with the compound.

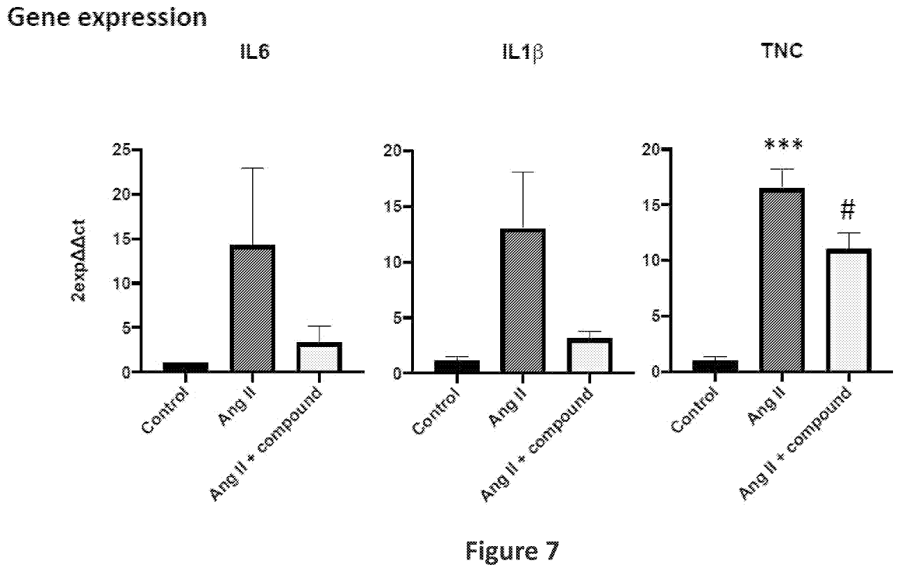

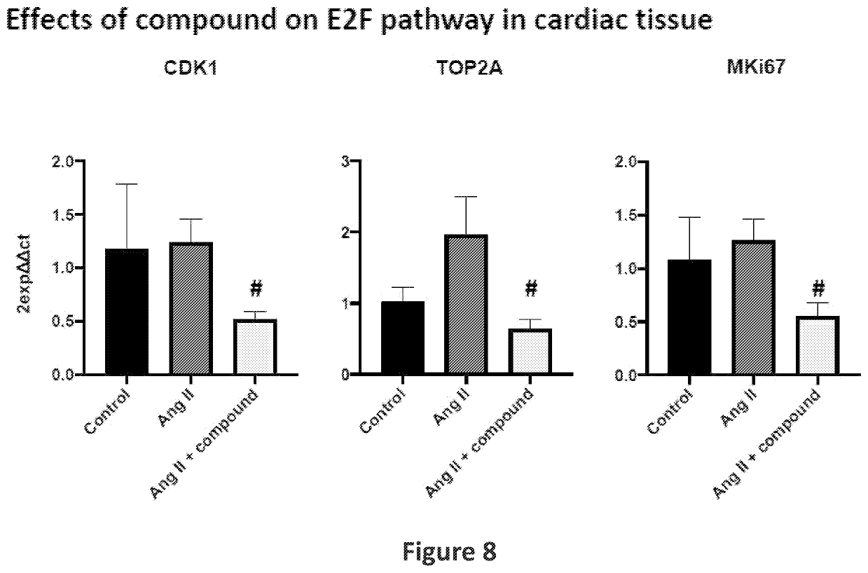

The expression of genes related with inflammatory and fibrotic processes were also investigated. Ang II infused mice had significantly higher expression levels of IL6, IL1β, and TNC in left ventricle compared with control mice. Such upregulation was significantly reduced in mice treated with the compound of Formula (VIII) (FIG. 7). Moreover, it was shown that the compound of Formula (VIII) clearly downregulated the expression of a set of genes of E2F pathway. Furthermore, the expression of the genes related to E2F pathway, such CDK1, TOP2A and MKi67, were analyzed and the treatment with the compound of the present disclosure confirmed its capacity to inhibit gene expression related with this hallmark (FIG. 8).

### Example 5: Real-Time Quantitative PCR

Oral treatment with the compound of Formula (VIII) induced a significant reduction of inflammatory and fibrotic markers to the control level (FIG. 7). In addition, treatment with the compound of Formula (VIII) induced a reduction of expression in genes related to E2F (FIG. 8).

Total RNA was isolated from mice frozen skin tissue using QIAzol lysis reagent (Qiagen, Hilden, Germany) and purified with RNeasy mini kit (Qiagen). Total RNA (1 μg) was retrotranscribed using the iScript™ cDNA Synthesis Kit (Bio-Rad) and the cDNA generated was analyzed by real-time PCR using the iQ™ SYBR Green Supermix (Bio-Rad) using a CFX96 Real-Time PCR Detection System (Bio-Rad). Real-time PCR was performed using a CFX96 Real-Time PCR Detection System (Bio-Rad). The GAPDH housekeeping gene was used to standardize the mRNA expression levels in every sample. Expression levels were calculated using the 2-ΔΔCt method. Sequences of oligonucleotide primers are given in Table 1. Gene expression were normalized to GADPH or mRNA levels in each sample and expressed using the 2-ΔΔCt method.

TABLE 1
List of mouse primer sequences used in
quantitative Polymerase Chain Reaction.

| | | |
|---|---|---|
| IL-6 | 5′-GAACAACGAT | 5′-TCCAGGTAGC |
| | GATGCACTTG | TATGGTACTC |
| | C-3′ | C-3 |
| | (SEQ ID NO: 1) | (SEQ ID NO: 2) |
| IL-1β | 5′-CTCCACCTCA | 5′-GCCGTCTTTC |
| | ATGGACAGAA | ATTACACAGG |
| | -3 | -3′ |
| | (SEQ ID NO: 3) | (SEQ ID NO: 4) |
| TNC | 5′-ATCCCTTCAT | 5′-GCATCCGTAC |
| | CAGCAGTCCA | CAAAACCATC |
| | -3′ | -3′ |
| | (SEQ ID NO: 5) | (SEQ ID NO: 6) |
| CDK1 | 5′-CAGAGATTG | 5′-GAAAGGTGTT |
| | ACCAGCTCT | CTTGTAGTCC |
| | T-3′ | -3′ |
| | (SEQ ID NO: 7) | (SEQ ID NO: 8) |
| TOP2A | 5′-CGGAATGACA | 5′-GCATTGTAAA |
| | AGCGAGAAGT | GATGTATCGT |
| | AA-3′ | GGAC-3′ |
| | (SEQ ID NO: 9) | (SEQ ID NO: 10) |
| Mki67 | 5′-AATCCAACTC | 5′-TTGGCTTGCT |
| | AAGTAAACGG | TCCATCCTCA |
| | GG-3′ | -3′ |
| | (SEQ ID NO: 11) | (SEQ ID NO: 12) |
| GAPDH | 5′-TGGCAAAGTG | 5′-AAGATGGTGA |
| | GAGATTGTTG | TGGGCTTCCC |
| | CC-3′ | G-3′ |
| | (SEQ ID NO: 13) | (SEQ ID NO: 14) |

### Example 6: Compound of Formula (VIII) Ameliorated Proliferation of Cardiac Myofibroblast by Regulation of E2F Pathway

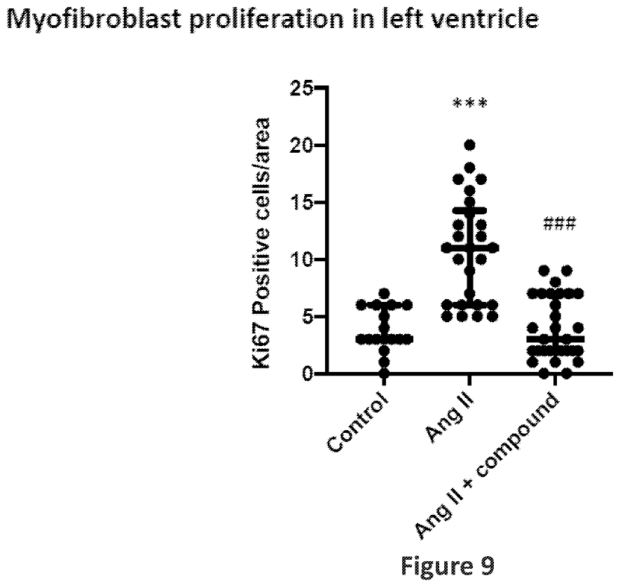

Proliferation of fibroblast is an important event of Ang II-induced fibrosis and plays an important role in cardiac fibrosis. Non-cardiomyocytes proliferation was demonstrated by staining with a mAb that identifies Ki 67 (FIG. 9). Ki 67 is a protein that is encoded by the MKI67 gene and is a cellular marker for proliferation. Cardiac tissue sections (5 μM-thick) were deparaffinized and boiled for 10 minutes in sodium citrate buffer (10 mM, pH 6.0) (Sigma-Aldrich, Madrid, Spain) for antigen retrieval. The sections were then incubated in a PBS washing buffer containing 0.1% Triton X-100 (Sigma-Aldrich, Madrid, Spain) three times for 10 minutes. Nonspecific binding was blocked with 3% bovine serum albumin (BSA) (Sigma-Aldrich, Madrid, Spain). The sections were incubated overnight at 4° C. with Ki 67 antibody (1:100 dilution, ab15580, Abcam, Cambridge, UK). After three washes for 10 minutes each in wash buffer, slides were incubated with secondary antibody anti-rabbit Texas Red (1:100 dilution, #A-6399, Thermo Fischer Scientific, Waltham, Mass., USA) for 1 hour at room temperature in the dark. The tissue sections were then mounted using Vectashield Antifade Mounting Medium with DAPI (H-1200, Vector Laboratories, Burlingame, Calif., USA) before the slides were washed 3 times for 10 minutes. All images were acquired using a spectral confocal laser-scanning microscope LSM710, (Zeiss, Jena, Germany) with a 63× Plan-Apochromat oil immersion lens and quantified in 10-15 randomly chosen fields using ImageJ software (rsb.info.nih.gov/ij/).

### Example 7: Preventive Effect of the Compound of Formula (VIII) on Renal Fibrosis Mouse Induced by Ang II

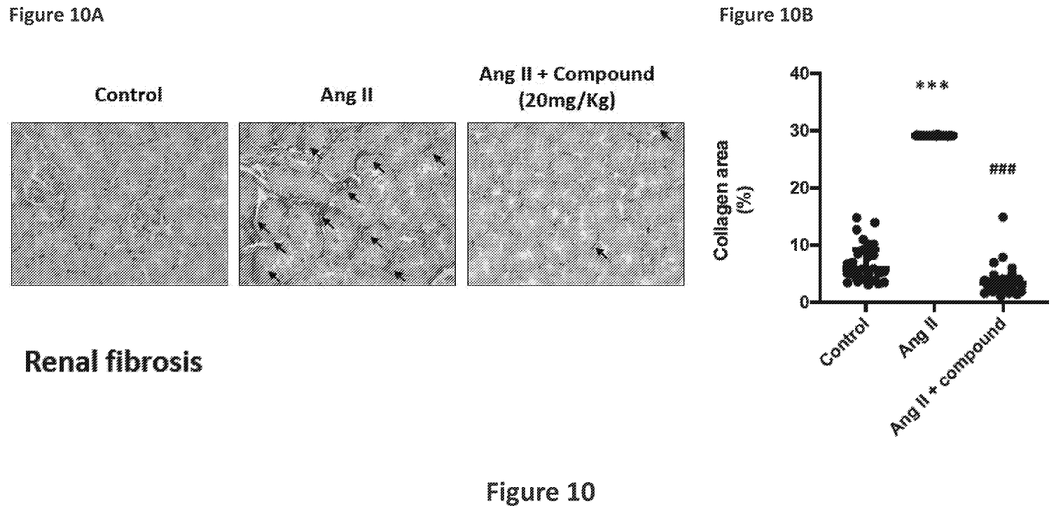

Ang II is the main peptide of the renin angiotensin system and a renal growth factor, inducing hyperplasia/hypertrophy depending on the cell type, and it induces renal fibrosis. Renal fibrosis was induced in mice by infusion of Ang II (1.0 mg kg⁻ min⁻) for two weeks. The treatment with the compound of Formula (VIII) by oral gavage was the same as describe in Example 2. After two weeks of treatment, the mice were sacrificed under ether anesthesia. Kidney samples were collected and fixed in 4% paraformaldehyde and embedded in paraffin. The paraffin sections (5 μm) were stained with Sirius Red for evaluation of fibrosis. Subcutaneous Ang II infusion induced a significant increase of collagen content and in the mice treated with compound, the induction of fibrosis in the kidney is prevented (FIG. 10).

### Example 8: Preventive Effect of the Compound of Formula (VIII) on Dermal Fibrosis Mouse Induced by Ang II

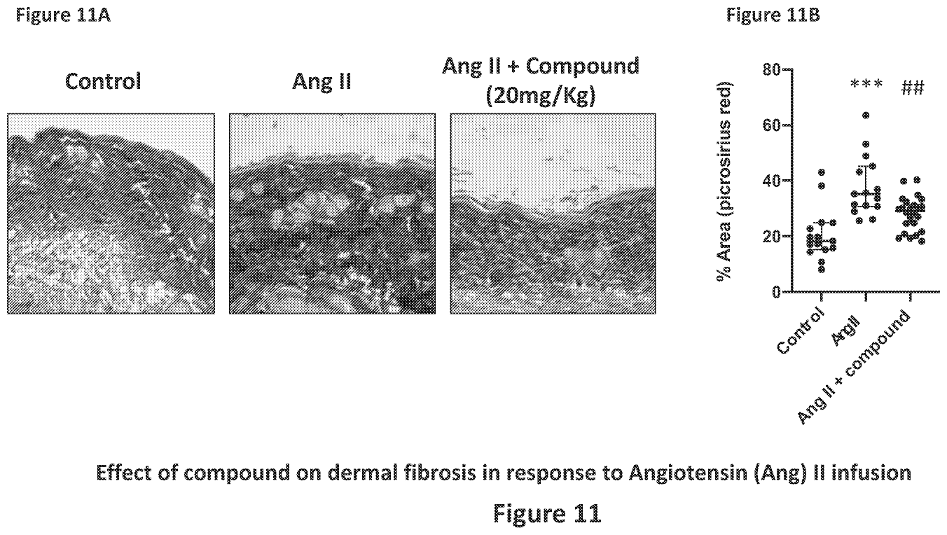

The local renin angiotensin system exists in almost all organs, including skin. Ang II induces fibrosis in the skin via accumulation of activated fibroblasts. Dermal fibrosis was induced in mice by infusion of Ang II (1.0 mg kg⁻ min⁻) for two weeks. The treatment with the compound of Formula (VIII) by oral gavage was the same as describe in Example 2. After two weeks of treatment, the mice were sacrificed under ether anesthesia. Skin samples were collected and fixed in 4% paraformaldehyde and embedded in paraffin. Skin sections (5 μm) were stained with Sirius Red for evaluation of fibrosis. Subcutaneous Ang II infusion induced a significant increase of collagen content and in the mice treated with compound, the induction of fibrosis in the skin is prevented (FIG. 11).

### Example 9: Therapeutic Effect of the Compound of Formula (VIII) on Cardiac and Renal Fibrosis Mouse Induced by Ang II

For the therapeutic model, infusion of Ang II for four weeks and the compound of Formula (VIII) (20 mg/kg) was administrated after two weeks of pump implantation. Subcutaneous infusion of Ang II using ALZET osmotic minipumps was used to study the preventive effects of the compound ALZET® osmotic minipumps (Model #2004, Charles Rives, Barcelona, Spain) delivering of Ang II (500 ng kg⁻¹ min⁻¹) (Sigma-Aldrich, Madrid, Spain) for four weeks. Compound treatment was daily administered in parallel, two weeks after the implantation of the subcutaneous pump, beneath the midscapular loose skin and consisted of daily oral gavage of compound of Formula (VIII) (20 mg/kg) or vehicle (Corn Oil/Maisine CC, 1/1). Mice were acclimated to manipulators for a week previously to the experiments in order to reduce stress and allow the detection of subtle changes in behavior by researchers. Mice weight was evaluated weakly and no significant changes were observed between experimental groups. No behavioral changes were observed during the protocols. Neither piloerection nor hunched appearances were appreciated during the experimental procedures. Mice were sacrificed by cervical dislocation and the heart, aorta, kidney, and lung were removed and processed for histological examination or were frozen for further analysis.

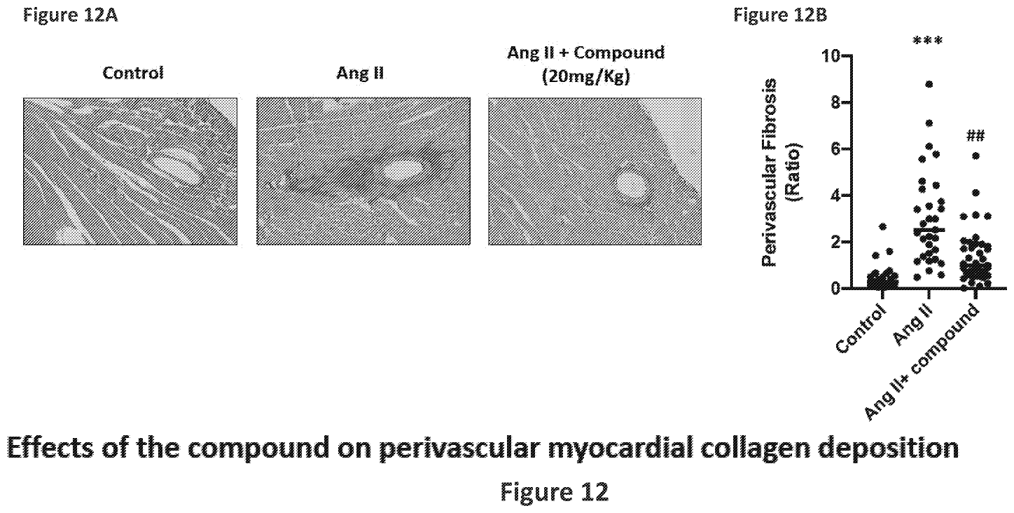

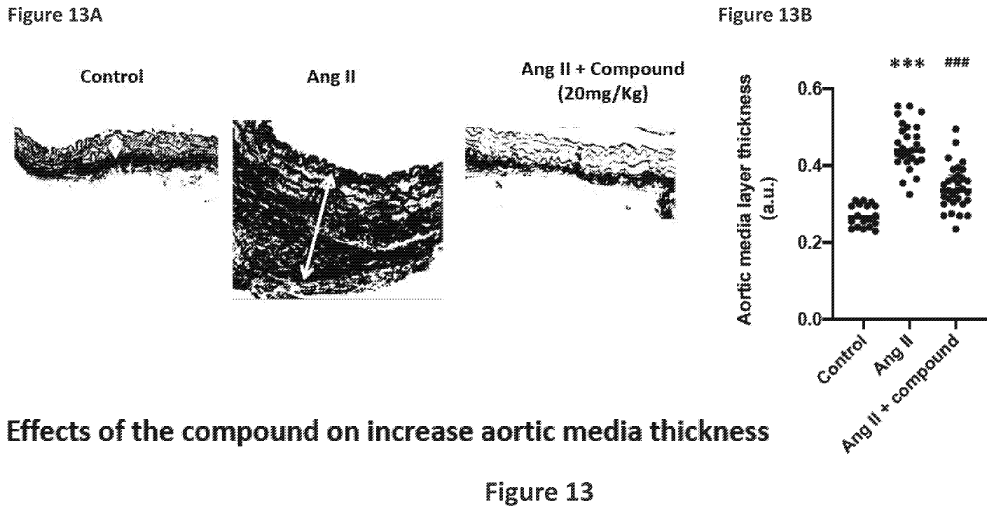

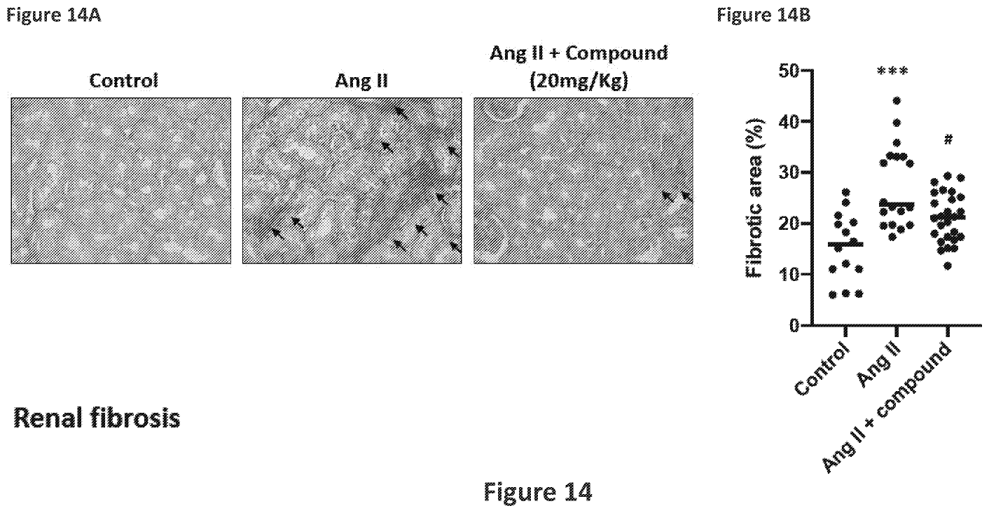

Ang II infusion for four weeks resulted in augmented on perivascular myocardial collagen deposition versus control and the compound reduces the collagen content around the vessels (FIG. 12). In addition, the Ang II administration in mice resulted in augmentation of aorta adventitia layer thickness, which was reduced by compound treatment (FIG. 13). Moreover, collagen deposition on interstitial renal tissue was augmented in mice infused with Ang II and was reduced by the treatment with the compound of the present disclosure (FIG. 14).

### Example 10: Therapeutic Effect of the Compound of Formula (VIII) on Pulmonary Fibrosis Induced by Ang II in Mice

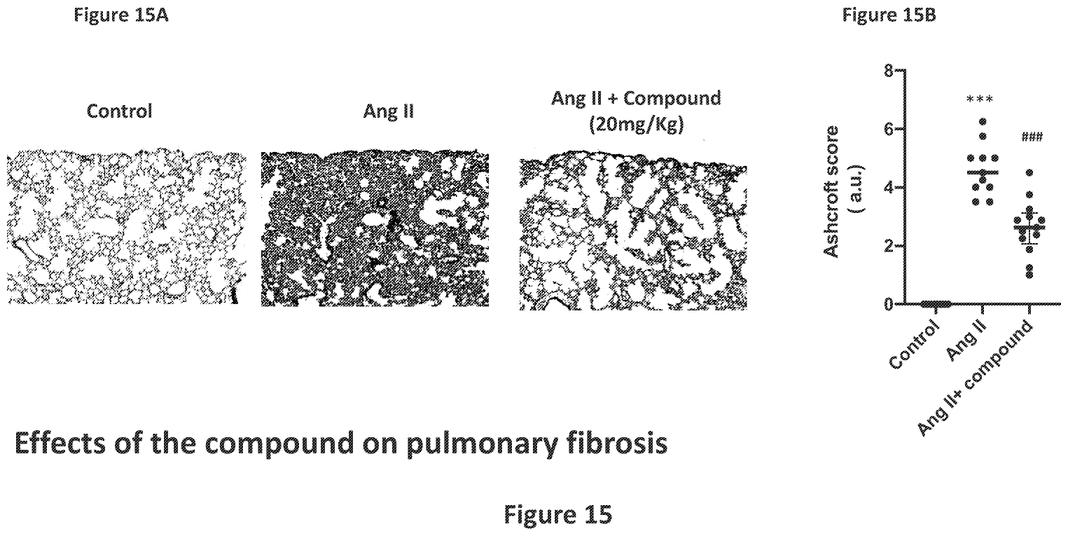

There is significant in vivo evidence suggesting that the angiotensin system is involved in lung fibrosis (Uhal B D et al., 2012, Int. J. Biochem. Cell Biol., 44:465-468). Lung fibrosis was induced in mice by infusion of Ang II (500 ng kg⁻ min⁻) for four weeks. The treatment with the compound of Formula (VIII) by oral gavage was the same as describe in Example 9. After two weeks of treatment, the mice were sacrificed under ether anesthesia. Lung samples were collected and fixed in 4% paraformaldehyde and embedded in paraffin. The paraffin sections (5 μm) were stained with Masson's trichrome (Merck Millipore, Darmstadt, Germany) for evaluation of fibrosis. Three-four random fields of each lung biopsy were photographed, digitalized using a Leica DFC420c camera and analyzed using Image J software in a blinded manner by two independent observers. The Ashcroft score was used to determine the degree of fibrosis in lung specimens as previously described (Ashcroft T et al., 1988, Clin. Pathol., 41:467-470; Hubner R H et al., 2008, Biotechniques, 44:507-511). Subcutaneous Ang II infusion induced pulmonary fibrosis which is prevented with compound (FIG. 15).

### Example 11: Effect of the Compound of Formula (VIII) (also called VCE-004.8) on Fibroblasts Differentiation into Myofibroblasts

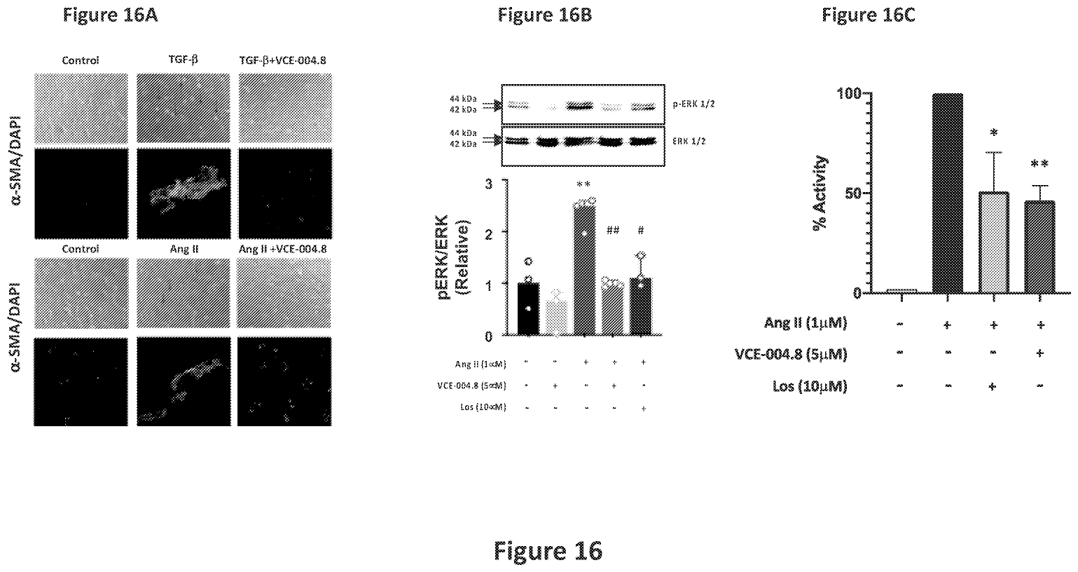

The profibrotic activity of Ang II on cardiac fibroblasts in vitro and in vivo has been extensively documented (H. H. Sigusch et al., 1996, Cardiovasc Res, 31:546-554). To study myofibroblast differentiation in vitro, immortalized cardiac fibroblasts (hICF) were stained with for α-SMA expression, a specific marker for myofibroblasts differentiation. Immortalized primary human cardiac fibroblasts (hICF) with SV40 Large T antigen were obtained from Innoprot SL (Derio, Spain) and cultured in low passages (<5 passages). The cells were maintained in a humidified atmosphere at 37° C. containing 5% CO₂, 10% fetal bovine serum (FBS) and 1% (v/v) penicillin/streptomycin in Fibroblast Medium-2 (P60108-2, Innoprot, Derio, Spain). Ang II was purchased from Sigma-Aldrich Co (A9525, Sigma, Madrid, Spain) and TGFb1 (Transforming Growth Factor-b)-1 (10 ng/mL) was obtained from ImmunoTools GmbH (Friesoythe, Germany). Both TGF-β and Ang II induced α-SMA protein expression in cultured hICF and pretreatment with compound of Formula (VIII) decreased α-SMA expression. The differentiation to myofibroblast was associated with morphological changes of cellular hypertrophy. Cell shape variations from spindle-shaped were observed and they exhibited a dendritic morphology compared to cells treated with Ang II or TGFb, which were more spread, hallmark of myofibroblast differentiation (FIG. 16A).

The effect of compound of Formula (VIII) on Ang II signaling pathways in hICF was investigated. ERK1+2 and NFAT activation are two pathways activated by Ang-II (Wakatsuki et al., 2004, Trends Biochem Sci, 29:609-617). hICF were preincubated with compound of Formula (VIII), treated with Ang II for 30 minutes, washed with PBS and proteins extracted in 50 μL of lysis buffer (50 mM Tris-HCl pH 7.5, 150 mM NaCl, 1% (v/v) NP-40, 10% (v/v) glycerol, 10 mM NaF, 1 mM Na₃VO₄, aprotinin (10 μg/ml), leupeptine (10 μg/ml), pepstatin (1 μg/ml) and 1 mM PMSF saturated). Separated proteins in 10% SDS/PAGE gels were transferred to PVDF membranes and blocked in TBS solution containing 0.1% Tween 20 and 5% bovine serum albumin (BSA) for 1 hour at room temperature. Immunodetection of specific proteins was carried out by incubation with primary antibodies against p-ERK (1:1000; #4695 Cell Signaling Technology) and ERK (1:1000, M5670, Sigma). Membranes were incubated with horseradish peroxidase-conjugated secondary antibody was added and proteins detected by chemiluminescence system (GE Healthcare Europe GmbH). Compound of Formula (VIII) inhibited Ang II-induced ERK1+2 phosphorylation (FIG. 16B).

hICF cells were transiently transfected for 24 hours with the Gal4-Luc reporter plasmid (which includes five Gal4 DNA binding sites fused to the luciferase gene) and the Gal4-NFAT₁₋₄₁₅plasmid for 24 hours using Roti-Fect (Carl Roth GmbH & Co. KG, Karlsruhe, Germany). Then cells were stimulated for 6 hours and luciferase activity in the cell lysates was measured. The specific transactivation expressed as a percentage of activation relative to control. Compound of Formula (VIII) inhibited Ang II-induced NFAT activation (FIG. 16B).

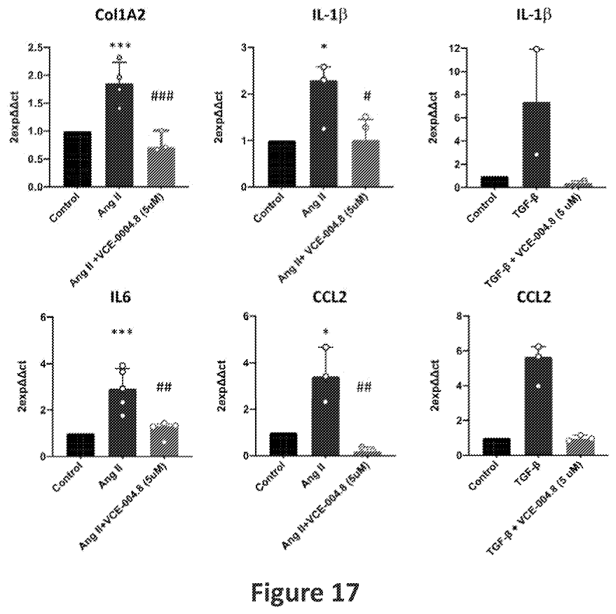

hICF (5×10⁴ cells/cm²) were preincubated with Compound of Formula (VIII), stimulated with Ang II for either 2 hours or 24 hours and total RNA was extracted using High Pure Isolation Kit (Roche Diagnostic, Switzerland). Total RNA (1 μg) was retrotranscribed using the iScript™ cDNA Synthesis Kit (Bio-Rad) and the cDNA analyzed using a CFX96 Real-Time PCR Detection System (Bio-Rad) by real-time PCR using the iQ™ SYBR Green Supermix (Bio-Rad). Human HPRT was used for in vitro studies to standardize the mRNA expression levels in each sample. Expression levels were calculated using the 2-ΔΔCt method. Sequences of oligonucleotide primers are shown in Table 2. Compound of Formula (VIII) inhibited the mRNA expression of IL1b, IL6, Col1A2 and CCL2, induced by Ang II or TGFb in hICF cells (FIG. 17).

TABLE 2
List of human primer sequences used in
quantitative Polymerase Chain Reaction.

| | | |
|---|---|---|
| Gene | Forward | Reverse |
| hIL1b | 5′-GACCTTCCAG | 5′-AGCTCATATG |
| | GATGAGGACA | GGTCCGACAG |
| | -3′ | -3′ |
| | (SEQ ID NO: 15) | (SEQ ID NO: 16) |
| hIL6 | 5′-GGTACATCCT | 5′-GTGCCTCTTT |
| | CGACGTGTCT | GCTGCTTTC |
| | -3′ | AC-3′ |
| | (SEQ ID NO: 17) | (SEQ ID NO: 18) |
| hCOL1A2A | 5′-CCGTGCTTCT | 5′-CTTGCCCC |
| | CAGAAGACAG | ATTCATTCAT |
| | -3′ | CA-3′ |
| | (SEQ ID NO: 19) | (SEQ ID NO: 20) |
| hCCL2 | 5′-CCCCAGTCAC | 5′-TGGAATCCTG |
| | CTGCTGTTAT | AACCC ACTTC-3′ |
| | -3′ | |
| | (SEQ ID NO: 21) | (SEQ ID NO: 22) |
| hHPRT | 5′-ATGGGAGGCC | 5′-ATGTAATCC |
| | ATCACATTGT | AGCAGGTCAGC |
| | -3′ | AA-3′ |
| | (SEQ ID NO: 23) | (SEQ ID NO: 24) |

It is to be understood that the Figures and descriptions of the present disclosure have been simplified to illustrate elements that are relevant for a clear understanding of the present disclosure, while eliminating, for the purpose of clarity, many other elements. Thus, those of ordinary skill in the art may recognize that other elements and/or steps are desirable and/or required in implementing the present disclosure. However, because such elements and steps are well known in the art, and because they do not facilitate a better understanding of the present disclosure, a discussion of such elements and steps is not provided herein. The disclosure herein is directed to all such variations and modifications to such elements and methods known to those skilled in the art.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1.-27. (canceled)

28. A method for preventing or treating a disease or disorder in a subject in need thereof caused by renin-angiotensin system (RAS) dysfunction and/or over-expression of angiotensin II and inflammation, wherein the method comprises administering a therapeutically amount of a compound having the structure of Formula (I) or a composition thereof to a subject in need thereof, 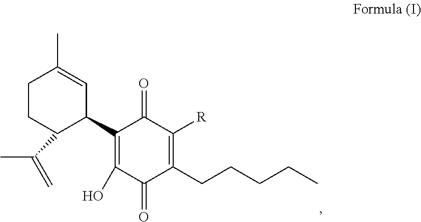
wherein R is the nitrogen atom of a group independently selected from a linear or branched alkylamine, an arylamine, an arylalkylamine, a heteroarylamine, a heteroarylalkylamine, a linear or branched alkenylamine, a linear or branched alkynylamine, or NH₂.

29. The method of claim 28, wherein the compound having the structure of Formula (I) is independently selected from the group consisting of: 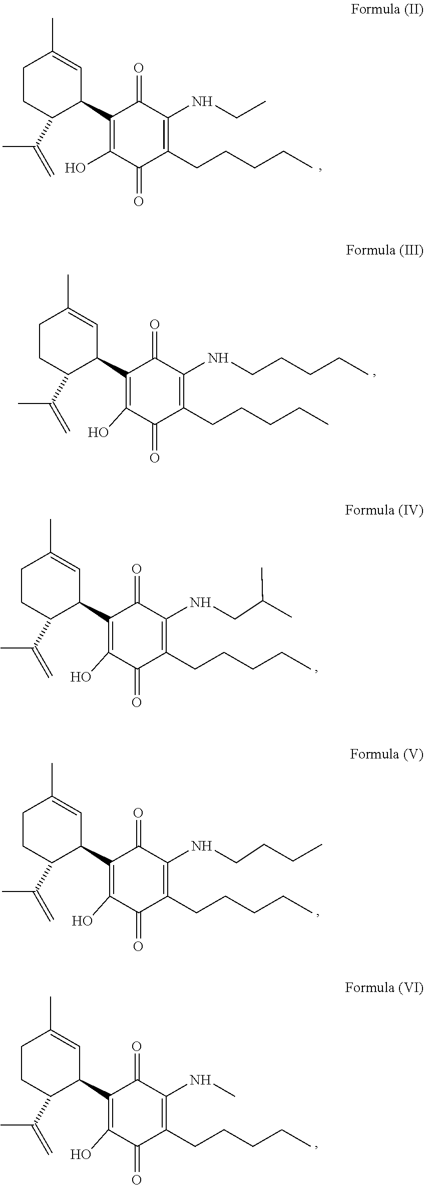 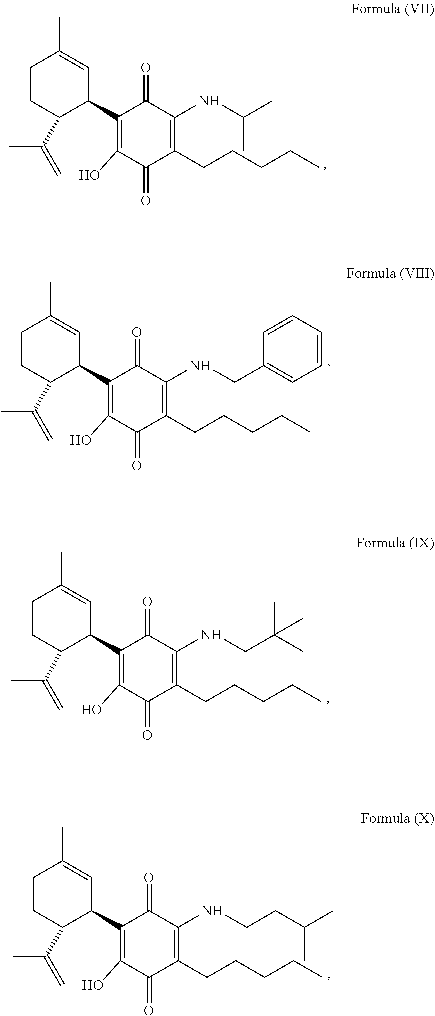
and any combination thereof.

30. The method of claim 28, wherein the compound having the structure of Formula (I) or a composition thereof inhibits one more of the activity of the E2F pathway, the activity of angiotensin II-induced ERK1/2 phosphorylation, the activity of angiotensin II-induced NFAT activation of at least one gene, proliferation of myofibroblasts, proliferation of fibroblasts, proliferation of fibrosis, macrophage accumulation, macrophage infiltration, collagen accumulation or deposition, development of aortic media thickness, development of aortic media thickness associated with fibrosis, activity of inflammatory response, activity of at least one fibrotic protein, activity of interferon response, activity of IFN-α response, activity of IFN-γ response, immune response associated with fibrosis, autoimmune response associated with fibrosis, inflammation associated with fibrosis, and any combination thereof.

31. The method of claim 29, wherein the compound having the structure of Formula (I)-(X) or a composition thereof modulates at least one the gene selected from the group consisting of a E2F-dependent gene, cyclin-dependent kinase 1 (CDK1) gene, topoisomerase 2-alpha (TOP2A) gene and Proliferation Ki-67 (MKi67) gene, Interleukin 6 (IL6) gene, Interleukin 1β (IL1β) gene, tenascin-C (TNC) gene, and any combination thereof.

32. The method of claim 30, wherein the fibrotic disease or disorder is selected from the group consisting of renal fibrosis, cardiac fibrosis, cardiovascular fibrosis, perivascular myocardial collagen accumulation or deposition, renal collagen accumulation or deposition, bronchiolitis obliterans organizing pneumonia (BOOP), acute respiratory distress syndrome (ARDS), asbestosis, accidental radiation induced lung fibrosis, therapeutic radiation induced lung fibrosis, sarcoidosis, silicosis, tuberculosis, Hermansky Pudlak syndrome, bagassosis, eosinophilic granuloma, Wegener's granulomatosis, lymphangioleiomyomatosis, cystic fibrosis, fatty liver disease, chronic graft-versus-host disease (cGVHD), sclerodermatous graft-versus-host disease, nephrogenic systemic fibrosis, Dupuytren's contracture, keloids, chronic graft rejection, scarring or wound healing abnormalities, post-operative adhesions, reactive fibrosis, disease or disorder associated with nephrotoxic agent exposure, disease or disorder associated with aminoglycoside exposure, disease or disorder associated with non-steroidal anti-inflammatory drug (NSAID) exposure, disease or disorder associated with immune-suppressant exposure, disease or disorder associated with nitrofurantoin exposure, disease or disorder associated with amiodarone exposure, disease or disorder associated with bleomycin exposure, disease or disorder associated with cyclophosphamide exposure, disease or disorder associated with methotrexate exposure, myocardial infarction, injury related tissue scarring, scarring associated with surgery, therapeutic radiation induced fibrosis, dermatomyositis (DM), disease or disorder associated with endothelial cell injury, such as autoimmune-based endothelial cell injury, and any combination thereof.

33. The method of claim 28, wherein the composition is a lipidic formulation comprising a lipid vehicle wherein the lipid vehicle is selected from the group consisting of Captex 300, Tween 85, Cremophor EL, Maisine 35-1, Maisine CC, Capmul MCM, corn oil, and any combination thereof.

34. The method of claim 33, wherein the lipid vehicle is an oil mixture comprising at least two oils.

35. The method of claim 34, wherein the oil mixture is a mixture of Maisine CC and maize oil.

36. A method for preventing or treating a fibrotic disease or condition caused by RAS dysfunction in a subject in need thereof, wherein the method comprises administering a therapeutically amount of a compound having the structure of the Formula (VIII) or a composition thereof: [chemical structure]
